(19) United States
(12) Patent Application Publication
(10) Pub. No.: US 2026/0355303 A1
(43) Pub. Date: Nov. 12, 2026

(54) COMPOSITIONS COMPRISING ENGINEERED ENVELOPES TO ENGAGE CD4 BINDING SITE BROADLY NEUTRALIZING ANTIBODY PRECURSORS

(71) Applicant: Duke University, Durham, NC (US)

(72) Inventors: James Counts, Durham, NC (US); Kevin O. Saunders, Durham, NC (US); Barton F. Haynes, Durham, NC (US)

(21) Appl. No.: 19/123,731

(22) PCT Filed: Oct. 24, 2023
(86) PCT No.: PCT/US2023/077669

§ 371 Date: Apr. 23, 2025

Related U.S. Application Data
(60) Provisional application No. 63/418,920, filed on Oct. 24, 2022.

(51) Int. Cl. A61K 39/21; A61K 39/385; C07K 14/005

(52) U.S. Cl. CPC A61K 39/21; A61K 39/385; C07K 14/005; C12N 2740/16134

(57) ABSTRACT

In certain aspects the invention provides HIV-1 immunogens, including HIV-1 envelopes with an optimized D loop, V5 loop, and/or CD4 binding loop for antibody induction.

This International Patent Application claims the benefit of and priority to LUS. Application No. 63/418,920, filed Oct. 24, 2022, entitled “Compositions Comprising Engineered Envelopes to Engage CID4 Binding Site Broadly Neutralizing Antibody Precursors,” the content of which is hereby incorporated by reference in its entirety.

This invention was made with government support under Center for HIV/AIDS Vaccine Immunology-Immunogen Design grant UM1-AI144371 from the NLH, MAID, Division of AIDS and HHS. The government has certain rights in the invention.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and method for induction of immune response, for example cross-reactive (broadly) neutralizing Ab (bNAb) induction.

In certain aspects, the invention provides a CH505 envelope immunogen comprising an optimized D loop, V5 loop, and/or CD4 binding loop. In certain aspects the invention provides CH505 TF envelopes comprising an optimized D loop. In certain aspects the invention provides CH505 T/F envelopes comprising an optimized V5 loop.

In certain aspects, the invention provides a recombinant protein or nucleic acid encoding a recombinant protein as described in Table 1. In certain aspects, the invention provides a selection of HIV-1 envelopes for use as prime and boost immunogens in methods to induce HIV-1 neutralizing antibodies. In certain aspects, the invention provides a selection of HIV-1 envelopes for use as boost immunogens in methods to induce HIV-1 neutralizing antibodies. In certain aspects, the invention provides one for more HIV-1 envelopes for use as a prime to induce HIV-1 neutralizing antibodies.

In certain embodiments, the invention provides a recombinant HIV-1 envelope sequence or nucleic acid encoding a recombinant protein HIV-1 envelope sequence comprising a mutation of one or more of amino acid residue R273, S274, N276, N279, 1277. T278, K282, 1284, R456, or M475. In certain embodiments, the mutation is N276G. In certain embodiments, the mutation is 1277P. In certain embodiments, the mutation is T278A. In certain embodiments, the mutation is T278R. In certain embodiments, the mutation is K282R. In certain embodiments, the mutation is R456Q. In certain embodiments, the mutation is R273S. In certain embodiments, the mutation is S274Q. In certain embodiments, the mutation is S274V. In certain embodiments, the mutation is N276K. In certain embodiments, the mutation is N279I. In certain embodiments, the mutation is N279L. In certain embodiments, the mutation is N279R. In certain embodiments, the mutation is I284W. In certain embodiments, the mutation is M475L In certain aspects, a recombinant HIV-1 envelope comprises any combination of mutations described herein. In certain aspects, a recombinant HIV-1 envelope comprises mutations N279H, K282R and R456Q. In certain aspects, a recombinant HIV-1 envelope comprises mutations N279H and K282R. In certain aspects, a recombinant HIV-1 envelope comprises mutations N279H and R456Q. In certain aspects, a recombinant HIV-1 envelope comprises mutations K282R and R456Q. In certain aspects, a recombinant HIV-1 envelope comprises mutations N279H, K282R, R456Q, and N276G. The amino acid numbering position is with respect to HXB2 envelope sequence.

In certain embodiments, the compositions contemplate nucleic acid, as DNA and/or RNA, or proteins immunogens either alone or in any combination. In certain embodiments, the methods contemplate genetic, as DNA and/or RNA, immunization either alone or in combination with envelope protein(s).

In certain embodiments the nucleic acid encoding an envelope is operably linked to a promoter inserted in an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides a nucleic acid sequence encoding any of the polypeptides of the invention, wherein the nucleic acid is operably linked to a promoter. In certain aspects the invention provides a nucleic acid consisting essentially of a nucleic acid sequence encoding any of the polypeptides of the invention, wherein the nucleic acid is operably linked to a promoter. In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain aspects the invention provides an expression vector consisting essentially a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In certain aspects the invention provides a composition comprising at least one nucleic acid encoding an HIV-1 envelope of the invention.

In certain embodiments, the compositions and methods employ an HIV-1 envelope as polypeptide instead of a nucleic acid sequence encoding the HIV-1 envelope. In certain embodiments, the compositions and methods employ an HIV-1 envelope as polypeptide, a nucleic acid sequence encoding the HIV-1 envelope, or a combination thereof. In certain embodiments, the polypeptides are recombinantly produced.

The envelope used in the compositions and methods of the invention can be a gp160, gp150, gp145, gp140, gp120, gp41, or N-termninal deletion variants thereof as described herein, cleavage resistant variants thereof as described herein, or codon optimized sequences thereof. In certain embodiments the composition comprises envelopes as trimers. In certain embodiments, envelope proteins are multimerized, for example trimers are attached to a particle such that multiple copies of the trimer are attached and the multimerized envelope is prepared and formulated for immunization in a human. In certain embodiments, the compositions comprise envelopes, including but not limited to trimers as particulate, high-density array on liposomes or other particles, for example but not limited to nanoparticles. In some embodiments, the trimers are in a well ordered, near native like or closed conformation. In some embodiments the trimer compositions comprise a homogenous mix of native like trimers. In some embodiments the trimer compositions comprise at least 65%, 70%, 75%, 80%, 85%, 90%, 95% native like trimers.

The polypeptide contemplated by the invention can be a polypeptide comprising any one of the polypeptides described herein. The polypeptide contemplated by the invention can be a polypeptide consisting essentially of any one of the polypeptides described herein. The polypeptide contemplated by the invention can be a polypeptide consisting of any one of the polypeptides described herein. In certain embodiments, the polypeptide is recombinantly produced. In certain embodiments, the polypeptides and nucleic acids of the invention are suitable for use as an immunogen, for example to be administered in a human subject.

In certain embodiments the envelope is any of the forms of HIV-1 envelope. In certain embodiments the envelope is a gp120, gp140, gp145 (i.e. with a transmembrane), gp150 envelope. In certain embodiments, gp140 is designed to form a stable trimer. In certain embodiments envelope protomers form a trimer which is not a SOSIP timer. In certain embodiment the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” which content is herein incorporated by reference in its entirety. In certain embodiments the envelopes of the invention are engineered and comprise non-naturally occurring modifications.

In certain embodiments, the envelope is in a liposome. In certain embodiments the envelope comprises a transmembrane domain with a cytoplasmic tail embedded in a liposome. In certain embodiments, the nucleic acid comprises a nucleic acid sequence which encodes a gp120, gp140, gp145, gp150, or gp160. In certain embodiments, where the nucleic acids are operably linked to a promoter and inserted in a vector, the vectors are any suitable vector. Non-limiting examples include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, ASO1 B, ASO1 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014), Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339).

In non-limiting embodiments, the adjuvant is an LNP. See e.g., without limitation Shirai et al. “Lipid Nanoparticle Acts as a Potential Adjuvant for Influenza Split Vaccine without Inducing Inflammatory Responses” Vaccines 2020, 8, 433; doi:10.3390/vaccines8030433, published 3 Aug. 2020. In non-limiting embodiments, LNPs used as adjuvants for protein compositions are composed of an ionizable lipid, cholesterol, lipid conjugated with polyethylene glycol, and a helper lipid. Non-limiting embodiment include LNPs without polyethylene glycol.

In certain aspects the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a stable pool of cells encoding any one of the envelopes of the invention suitable for recombinant expression.

In certain aspects, the invention provides a recombinant HIV-1 envelope polypeptide listed in Table 1 and/or FIG. 1 or 18. In certain embodiments, the polypeptide is a non-naturally occurring protomer designed to form an envelope trimer. The invention also provides nucleic acids encoding these recombinant polypeptides. Non-limiting examples of amino acids and nucleic acids of such protomers are shown in FIG. 1.

In certain aspects the invention provides a recombinant trimer comprising three identical protomers of an envelope from Table 1 and/or FIG. 1 or 18. In certain aspects the invention provides an immunogenic composition comprising the recombinant trimer and a carrier, wherein the trimer comprises three identical protomers of an HIV-1 envelope listed in Table 1 and/or FIG. 1 or 18. In certain aspects the invention provides an immunogenic composition comprising a nucleic acid encoding these recombinant HIV-1 envelope and a carrier.

In certain aspects the invention provides nucleic acids encoding HIV-1 envelopes for immunization wherein the nucleic acid encodes a gp120 envelope, gp120D8 envelope, a gp140 envelope (gp140C, gp140CF, gp140CFI) as soluble or stabilized protomer of a SOSIP trimer, a gp145 envelope, a gp150 envelope, or a transmembrane bound envelope.

In certain aspects the invention provides a selection of HIV-1 envelopes for immunization wherein the HIV-1 envelope is a gp120 envelope or a gp120D8 variant. In certain embodiments a composition for immunization comprises protomers that form stabilized SOSIP trimers.

In certain embodiments, the compositions for use in immunization further comprise an adjuvant.

In certain embodiments, wherein the compositions comprise a nucleic acid, the nucleic acid is operably linked to a promoter, and could be inserted in an expression vector.

In one aspect the invention provides a composition for a prime boost immunization regimen comprising any of envelopes described herein, or any combination thereof wherein the envelope is a prime or boost immunogen. In certain embodiments, the composition for a prime boost immunization regimen comprises one or more envelopes described herein, wherein the polypeptide is a non-naturally occurring protomer designed to form an envelope trimer, wherein the envelope is a prime or boost immunogen. In one aspect the invention provides a composition for a prime boost immunization regimen comprising one or more envelopes of the invention. In one aspect the invention provides a composition for a prime immunization comprising one or more envelopes of the invention.

In certain aspects the invention provides methods of inducing an immune response in a subject comprising administering a composition comprising a polypeptide and/or any suitable form of a nucleic acid(s) encoding an HIV-1 envelope(s) in an amount sufficient to induce an immune response.

In certain embodiments, the nucleic acid encodes a gp120 envelope, gp120D8 envelope, a gp140 envelope (gp140C, gp140CF, gp140CFI) as soluble or stabilized protomer of a SOSIP trimer, a gp145 envelope, a gp150 envelope, or a transmembrane bound envelope. In certain embodiments, the polypeptide is gp120 envelope, gp120D8 envelope, a gp140 envelope (gp140C, gp140CF, gp140CFI) as soluble or stabilized protomer of a SOSIP trimer, a gp145 envelope, a gp150 envelope, or a transmembrane bound envelope.

In certain embodiments, the methods comprise administering an adjuvant. In certain embodiments, the methods comprise administering an agent which modulates host immune tolerance. In certain embodiments, the administered polypeptide is multimerized in a liposome or nanoparticle. In certain embodiments, the methods comprise administering one or more additional HIV-1 immunogens to induce a T cell response. Non-limiting examples include gag, nef, pol, etc.

In certain aspects, the invention provides a recombinant HIV-1 envelope sequence or nucleic acid encoding a recombinant protein HIV-1 envelope sequence comprising a mutation of amino acid residue R273, S274, N276, N279, I277, T278, K282, 1284, R456, or M475 (including, but not limited to, N276G, I277P, T278A, T278R, K282R, R456Q, R273S, 5274Q, S274V, N276K, N279H, N279L, N279R, 1284W, or M475L). In certain aspects, a recombinant HIV-1 envelope or nucleic acid encoding a recombinant protein HIV-1 envelope sequence comprises mutations N279H, K282R and R456Q. In certain aspects, a recombinant HIV-1 envelope or nucleic acid encoding a recombinant protein HIV-1 envelope sequence comprises mutations N279H and K282R. In certain aspects, a recombinant HIV-1 envelope or nucleic acid encoding a recombinant protein HIV-1 envelope sequence comprises mutations N279H and R456Q. In certain aspects, a recombinant HIV-1 envelope or nucleic acid encoding a recombinant protein HIV-1 envelope sequence comprises mutations K282R and R456Q. In certain aspects, a recombinant HIV-1 envelope or nucleic acid encoding a recombinant protein HIV-1 envelope sequence comprises mutations N279H, K282R, R456Q, and N276G. The amino acid numbering position is with respect to HXB2 envelope sequence. Non-limited examples of envelopes are listed in Table 1 and FIG. 1 or 18. In some embodiments, the amino acid sequence of one monomer comprised in the trimer is shown in FIG. 1 or 18. In some embodiments, the trimer is immunogenic. In some embodiments the trimer binds to any one of the antibodies PGT145, PGT151, CH235UCA, (1235, 1-18, 2G12, or any combination thereof. In some embodiments the trimer does not bind to antibody 1913 and/or I7B.

In certain aspects, the invention provides a pharmaceutical composition comprising any one of the recombinant trimers of the invention. In certain embodiments the compositions comprising trimers are immunogenic. The percent trimer in such immunogenic compositions could vary. In some embodiments the composition comprises 70%, 71%, 72%, 73%, 74%, 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, or 99% stabilized trimer.

In certain embodiments, the recombinant protein nanoparticle comprises the envelope and ferritin. In certain embodiments, the inventive designs comprise modifications, including without limitation linkers between the envelope and ferritin designed to optimize ferritin nanoparticle assembly.

In certain aspects, the invention provides a composition comprising any one of the inventive envelopes or nucleic acid sequences encoding the same.

In certain aspects, the invention provides compositions comprising a nanoparticle which comprises any one of the envelopes of the invention.

In certain embodiments, the nanoparticle is a ferritin self-assembling nanoparticle.

In certain aspects, the invention provides a method of inducing an immune response in a subject comprising administering an immunogenic composition comprising any one of the envelopes of the invention. In certain embodiments, the composition is administered as a prime and/or a boost. In certain embodiments, the composition comprises nanoparticles. In certain embodiments, methods of the invention further comprise administering an adjuvant.

In certain aspects, the invention provides a composition comprising a plurality of nanoparticles comprising a plurality of the envelopes/trimers of the invention. In non-limiting embodiments, the envelopes/trimers of the invention are multimeric when comprised in a nanoparticle. The nanoparticle size is suitable for delivery. In non-liming embodiments the nanoparticles are ferritin-based nanoparticles.

In certain aspects, the invention provides nucleic acids comprising sequences encoding polypeptides or proteins of the invention. In certain embodiments, the nucleic acids are DNAs. In certain aspects, the invention provides expression vectors comprising the nucleic acids of the invention.

In certain aspects, the invention provides a recombinant HIV-1 envelope polypeptide according to Table 1 and/or FIG. 1 or 18. In certain embodiments, the polypeptide is a non-naturally occurring protomer. In some embodiments, the polypeptide is designed to form an envelope trimer. In certain embodiments, the envelope is based on CH505 T/F envelope and comprises optimized sequence for binding to CH235 lineage members, including without limitation CH235 UCA. In certain embodiment, the envelope comprises a mutation at position R273, S274, N276, N279, I277, T278, K282, 1284, R456 or M475, or a combination thereof (including, but not limited to, N276G, I277P, T278A, T278R, K282R, R456Q, R273S, S274Q, S274V, N276K, N279H, N279L, N279R, f284W, or M475L). In certain aspects, a recombinant HIV-1 envelope comprises mutations N279H, K282R and R456Q. In certain aspects, a recombinant HIV-1 envelope comprises mutations N279H and K282R. In certain aspects, a recombinant HIV-1 envelope comprises mutations N279H and R456Q. In certain aspects, a recombinant HIV-1 envelope comprises mutations K282R and R456Q. In certain aspects, a recombinant HIV-1 envelope comprises mutations N279H, K282R, R456Q, and N276G. The amino acid numbering position is with respect to HXB2 envelope sequence. In certain embodiments the envelope polypeptide is designed to multimerize. In some embodiments the envelope sequence comprises a self-assembling protein. In certain embodiments, the self-assembling protein is a ferritin. In other embodiments, the self-assembling protein is added via a sortase A reaction. In certain embodiments, the modifications described herein can be incorporated into any CH505 based envelope.

In some aspects, the invention provides a recombinant trimer comprising three identical protomers of an envelope from Table 1 and/or FIG. 1. In some embodiments, the invention provides an immunogenic composition comprising the recombinant trimer and a carrier, wherein the trimer comprises three identical protomers of an HIV-1 envelope listed in Table 1 and/or FIG. 1.

In some embodiments, the invention provides an immunogenic composition comprising a nucleic acid encoding the recombinant HIV-envelope and a carrier. In some embodiments, the envelopes are or are designed as trimers, and/or nanoparticles.

In some embodiments the immunogenic composition further comprises an adjuvant.

In some embodiments, the nucleic acid encoding one or more envelope selected from FIG. 1 or 18 any combination thereof is operably linked to a promoter. In some embodiments, the nucleic acid is inserted in an expression vector.

In some aspects, the invention provides a method of inducing an immune response in a subject comprising administering a composition comprising any suitable form of a nucleic acid(s) encoding one or more envelope selected from Table 1 or FIG. 1 or 18 or any combination thereof in an amount sufficient to induce an immune response.

In some embodiments, the composition administered comprises a nucleic acid encoding a gp120 envelope, gp120DS envelope, a gp140 envelope (gp140C, gp140CF, gp140CFI) as soluble or stabilized protomer of a SOSIP trimer, a gp145 envelope, a gp150 envelope, a transmembrane bound envelope, a gp160 envelope or an envelope designed to multimerize.

In some embodiments, the composition administered comprises a polypeptide, wherein the polypeptide is gp120 envelope, gp120D8 envelope, a gp140 envelope (gp140C, gp140CF, gp140CFI) as soluble or stabilized protomer of a SOSIP trimer, a gp145 envelope, a gp150 envelope, a transmembrane bound envelope, or an envelope designed to multimerize.

In some embodiments, the composition administered further comprises an adjuvant.

In some embodiments, the method further comprises administering an agent which modulates host immune tolerance. In some embodiment, the polypeptide administered is multimerized in a liposome or nanoparticle.

In some embodiments, the method further comprising administering one or more additional HIV-1 immunogens to induce a T cell response.

In some aspects, the invention provides a composition comprises a nanoparticle and a carrier, wherein the nanoparticle comprises an envelope, wherein the envelope is selected from Table 1 and/or FIG. 1 or 18 or any combination thereof. In some embodiments, the compositions comprises two, three, four or more different immunogens. In some embodiments the immunogens target different CH235 lineage members. In some embodiments, the immunogens target the CH235 lineage UCA. In non-limiting embodiments the different immunogens are selected from the various envelope designs described herein.

In some embodiments, the nanoparticle of the composition is ferritin self-assembling nanoparticle.

In some aspects, the invention provides a composition comprising a nanoparticle and a carrier, wherein the nanoparticle comprises a nucleic acid encoding the recombinant HIV-1 envelope polypeptide from Table 1 and/or FIG. 1 or 18.

In some embodiments, the nanoparticle of the composition is a ferritin self-assembling nanoparticle.

In some embodiments, the nanoparticle of the composition comprises multimers of trimers.

In some embodiments, the nanoparticle of the composition comprises 1-8 trimers.

In some aspects, the invention provides a method of inducing an immune response in a subject comprising administering an immunogenic composition comprising any one of the recombinant envelopes or compositions described herein. In some embodiments the methods comprise administering two, three, four or more different immunogens. In some embodiments, the different immunogens target different CH235 lineage members. In some embodiments, the different immunogens target the CH235 lineage UCA or CH235 intermediate antibodies. In non-limiting embodiments the different immunogens are selected from the envelope designs described herein—Tables 1 and/or FIG. 1 or 18.

In certain embodiments, the subject is infected with HIV (e.g., HIV-1). In certain embodiments, the subject is an HIV-uninfected individual. In certain embodiments, the subject is an HIV-infected individual. In certain embodiments, the administration to the HIV-infected individual induces broadly neutralizing antibodies. In certain embodiments the broadly neutralizing antibodies of the HIV-infected individual mediates viral (e.g., HIV-1) clearance from blood and tissues.

In some embodiments, the composition is administered as a single prime or as repetitive immunization prime. In preferred embodiments, the repetitive immunization is administered 3 or 4 times.

In some embodiments, the composition is administered as a single boost or as a repetitive series of boosts. In preferred embodiments, the repetitive series of boosts is administered 3 or 4 times.

In some embodiments, the composition is a first composition administered as a prime. In some embodiments, the composition is a second composition administered as one or more boosts. In some embodiments, the method comprises administering the first composition as a prime and administering the second composition as one or more boosts. In preferred embodiments, the first composition and the second composition are different.

In some aspects, the invention provides a nucleic acid encoding any of the recombinant envelopes described herein. In some embodiments, the invention provides a composition comprising the nucleic acid and a carrier.

In some embodiments, the invention provides a method of inducing an immune response in a subject comprising administering an immunogenic composition comprising the nucleic acid encoding any of the recombinant envelopes described herein. In some embodiments, the immunogenic composition further comprises a carrier.

In certain aspects, the invention provides an immunogenic composition or composition, wherein the composition comprises at least two different HIV-1 envelope polypeptides or nucleic acids encoding a recombinant HIV-1 envelope polypeptide, or a combination thereof.

In certain aspects, the invention provides an immunogenic composition comprising a first immunogen and a second immunogen, wherein the first immunogen is a recombinant HIV-1 envelope polypeptide from Table 1 and/or FIG. 1 or 18, or encoded by a nucleic acid encoding said recombinant HIV-1 envelope polypeptide, and wherein the second immunogen is a different recombinant HIV-1 envelope polypeptide from Table 1 and/or FIG. 1 or 18 or a nucleic acid encoding said different recombinant HIV-1 envelope polypeptide. In certain aspects, the invention provides a method of inducing an immune response in a subject comprising administering the immunogenic composition in an amount sufficient to induce an immune response. In certain embodiments, the method further comprising administering an agent which modulates host immune tolerance.

In certain embodiments, at least one of the first immunogen and the second immunogen is a recombinant HIV-1 envelope polypeptide. In certain embodiments, at least one of the first immunogen and the second immunogen is a recombinant trimer comprising three identical protomers of the recombinant HIV-1 envelope polypeptide. In certain embodiments, the first immunogen and the second immunogen are a recombinant HIV-1 envelope polypeptide. In certain embodiments, at least one of the first immunogen and the second immunogen is a nucleic acid. In certain embodiments, the first immunogen and the second immunogen are a nucleic acid.

In certain embodiments, the HIV-1 envelopes are in the form of a recombinant HIV-1 envelope polypeptides or nucleic acid, or a combination thereof. In certain embodiments, one or more of the HIV-1 envelopes is a recombinant trimer comprising three identical protomers of the recombinant HIV-1 envelope polypeptide. In certain embodiments, the composition comprises a carrier. In certain embodiments, the composition further comprises an adjuvant.

### BRIEF DESCRIPTION OF THE DRAWINGS

The patent or application file contains at least one drawing executed in color. To conform to the requirements for PCT patent applications, many of the figures presented herein are black and white representations of images originally created in color.

FIG. 1 shows non-limiting embodiments of amino acid sequences of envelopes of the invention. Signal peptide is underlined—the signal peptide is MPMGSLQPLATLYLLGiMLVASVLA (SEQ ID NO: I). Avi-tag is boxed—Avi-tag is GGGGSGLNDIEAQKIEWHE (SEQ ID NO: 2). FIG. 1 discloses SEQ ID NOS 10-24, respectively, in order of appearance.

FIG. 2 shows study aims.

FIG. 3 shows mutagenized library construction. FIG. 3 discloses SEQ ID NOS 25-28, respectively, in order of appearance.

FIG. 4 shows successive enrichment of SHM7 binding-enhanced mutants.

FIG. 5 shows that sequential selection of envelope variants enriched primarily D-Loop mutants.

FIG. 6 shows affinity of CH505 mutants (Bio-Layer Interferometry).

FIG. 7 shows that most mutations enhanced binding to CH235 lineage members.

FIG. 8 shows additional study aims.

FIG. 9 shows successive enrichment of UCA binding-enhanced mutants.

FIG. 10 shows that sequential selection of envelope variants again enriched primarily D-loop mutants.

FIG. 11 shows evolution of CH235.UCA-sorted mutants.

FIG. 12 shows methodology of creating site-scanning saturation library.

FIG. 13 shows enrichments of CH235.SHM7 binding-enhanced mutants.

FIG. 14 shows enrichments of CH235.UCA binding-enhanced mutants.

FIG. 15 shows mutation enrichment data.

FIG. 16 shows evaluation of CH235.SHM7-sorted mutants.

FIG. 17A-B show evaluation of CH235.1CA-sorted mutants,

FIG. 18 shows non-limiting embodiments of nucleic acid sequences of envelopes of the invention. Signal peptide is underlined. Avi-tag is boxed. FIG. 18 discloses SEQ ID NOS 29-45, respectively, in order of appearance.

FIG. 19A-C. FIG. 19A shows that SHM7 heterologous reactivity requires subset of mutations. FIG. 19B-C show that SHM7-selected double/triple mutants of CH505 place pressure on critical SHM7 mutations.

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (immunol. Rev. 254: 225-244, 2013). BnAbs are protective in rhesus macaques against SHIV challenge, but as yet, are not reproducibly induced by current vaccines.

For the past 25 years, the HV vaccine development field has used single or prime boost heterologous Envs as immunogens, but to date has not found a regimen to induce high levels of bnAbs.

Recently, a new paradigm for design of strategies for induction of broadly neutralizing antibodies was introduced, that of B cell lineage immunogen design (Nature Biotech. 30: 423, 2012) in which the induction of bnAb lineages is recreated. It was recently demonstrated the power of mapping the co-evolution of bnAbs and founder virus for elucidating the Env evolution pathways that lead to bnAb induction (Nature 496: 469, 2013).

### HIV-1 Envelopes

Described herein are nucleic acid and amino acid sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41-named as gp140ΔCFI (gp140CFI), gp140 Envs with the deletion of only the cleavage (C) site and fusion (F) domain—named as gp140ΔCF (gp140CF), gp140 Envs with the deletion of only the cleavage (C)—named gp140ΔC (gp140C) (See e.g. Liao et al. Virology 2006, 353, 268-282), gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

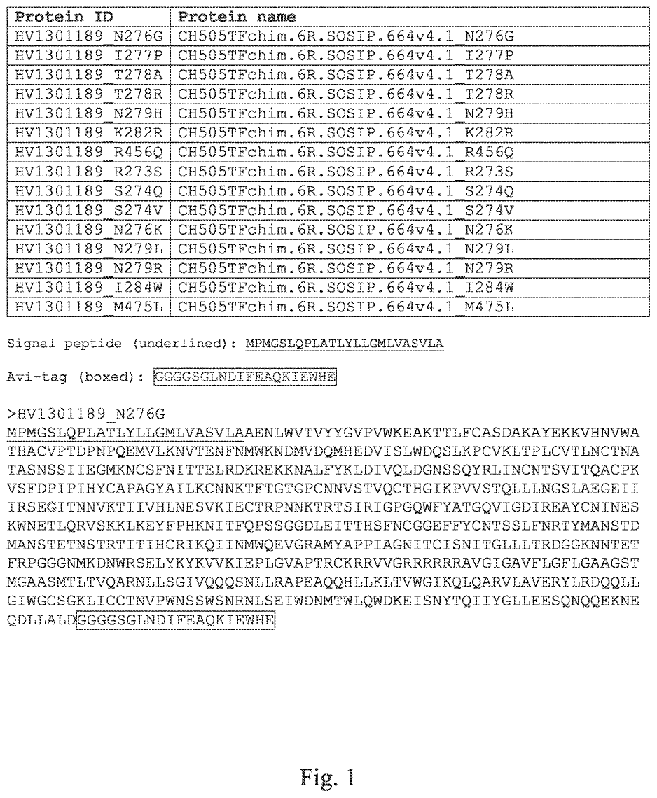

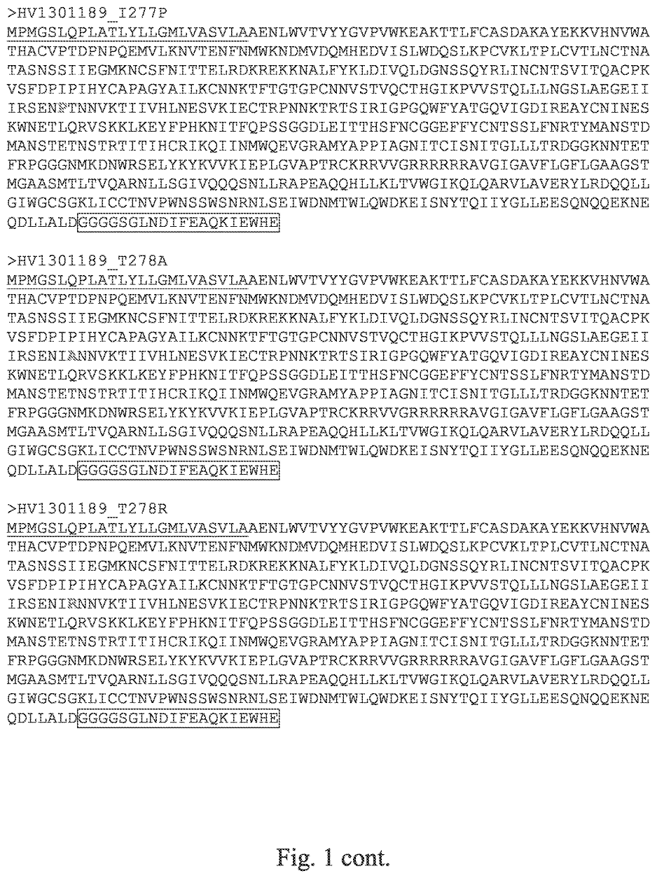

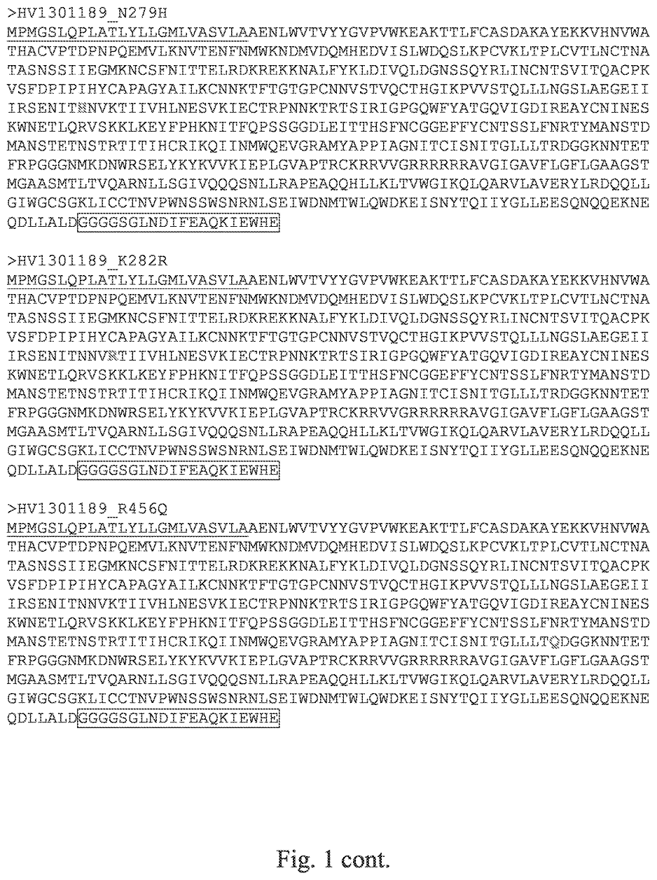

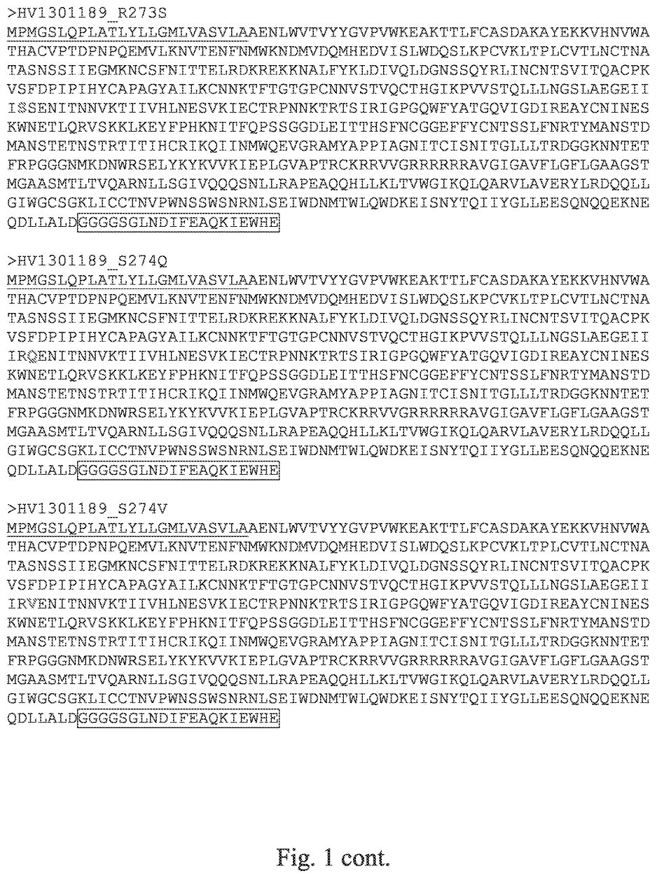

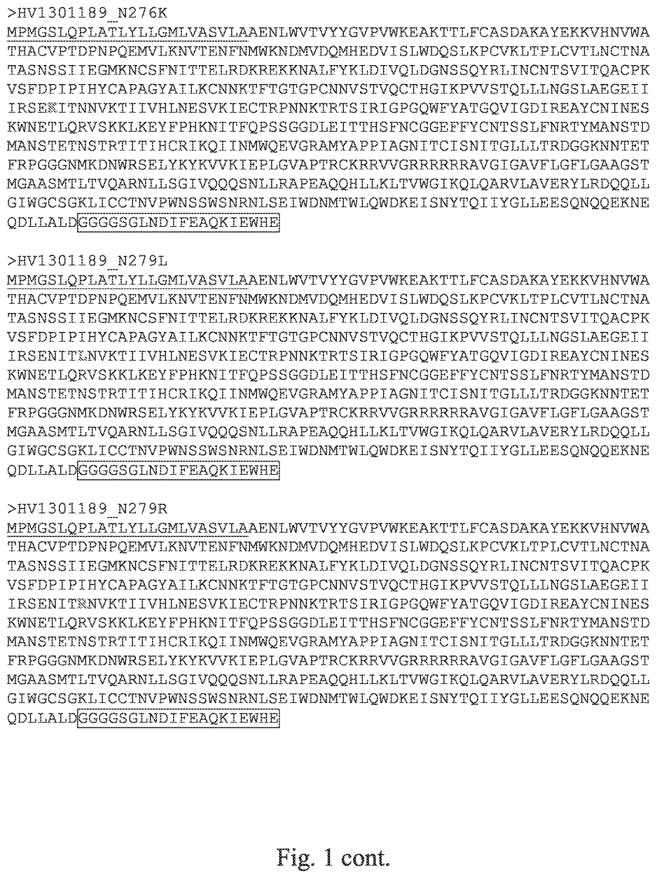

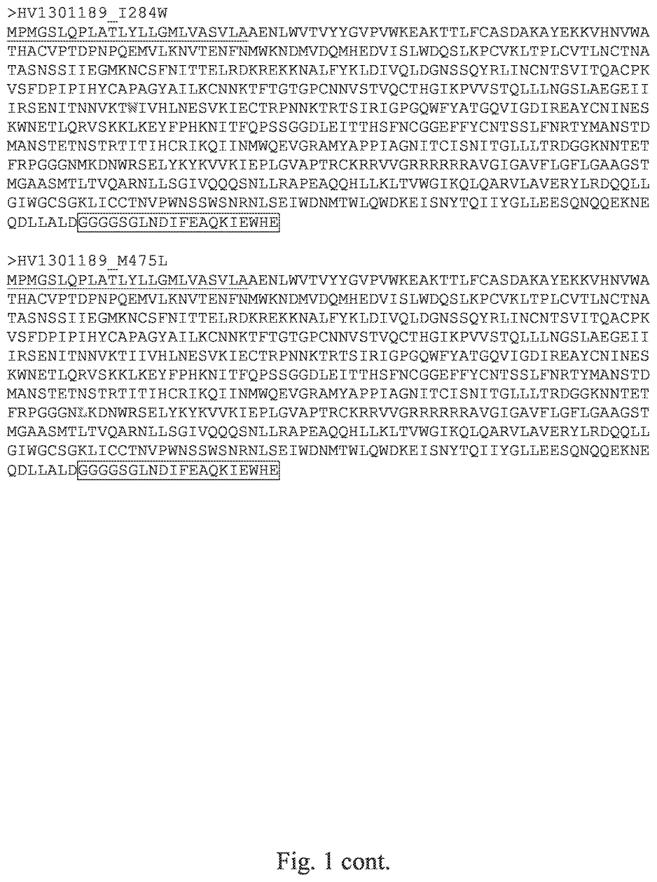

For example, it is well known in the art that during its transport to the cell surface, the gp160 polypeptide is processed and proteolytically cleaved to gp120 and gp4l proteins. Cleavages of gp160 to gp120 and gp41 occurs at a conserved cleavage site “REKR” (SEQ ID) NO: 3). See Chakrabarti et al. Journal of Virology vol. 76, pp. 5357-5368 (2002) see for example FIG. 1, and second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

The role of the furin cleavage site was well understood both in terms of improving cleave efficiency, see Binley et al. supra, and eliminating cleavage, see Bosch and Pawlita, Virology 64 (5):2337-2344 (1990); Guo et al. Virology 174: 217-224 (1990); McCune et al. Cell 53:55-67 (1988); Liao et al. J Virol. April; 87(8)4185-201 (2013).

Likewise, the design of gp140 envelope forms is also well known in the art, along with the various specific changes which give rise to the gp140C (uncleaved envelope), gp140CF and gp40CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR (SEQ ID NO: 4) is changed to ERVVEREKE (SEQ ID NO: 5), and is one example of an uncleaved gp140 form. Another example is the gp140C form which has the REKR site (SEQ ID NO: 3) changed to SEKS (SEQ ID NO: 6). See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol 353(2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . ”. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDA KAYEKEVINVWATHACVPTDPNPQE . . . (rest of envelope sequence is indicated as “ . . . ”) (SEQ ID NO: 7). In other embodiments, the delta N-design described for (CH505 T/F envelope can be used to make delta N-designs of other CH505 envelopes. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See U.S. Pat. No. 10,040,826, e.g. at pages 10-12, the contents of which is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain aspects, the invention provides composition and methods which CH505 Envs, as gp120s, gp140s cleaved and uncleaved, gp145s, gpl50s and gpl60s, stabilized and/or multimerized trimers, as proteins, DNAs, RNAs, or any combination thereof, administered as primes and boosts to elicit immune response. CH505 Envs as proteins would be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic (Gag group M consensus genes, are administered in a suitable vector, for example but not limited to HSV2, would be administered with each immunization with Env gene inserts in a suitable vector, for example but not limited to HSV-2.

### Nucleic Acid Sequences

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16: 319-23, 2010), recombinant mycobacteria (e.g. rBCG or M smegmatis) (Yu, J S et al. Clinical Vaccine Immunol. 14: 886-093,2007; ibid 13: 1204-11,2006), and recombinant vaccinia type of vectors (Santra S. Nature Med. 16: 324-8, 2010), for example but not limited to ALVAC, replicating (Kibler K V et al., PLoS One 6: e25674, 2011 nov 9.) and non-replicating (Perreaur M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54j 115-121; Arnaoty et al., Chapter 17 in Yves Bigot (ed.), Mobile Genetic Elements: Protocols and Genomic Applications, Methods in Molecular Biology, vol. 859, pp293-305 (2012); Arnaoty et al. (2013) Mol Genet Genomics. 2013 August; 288(7-8). 347-63. Nanocarrier technologies called Nanotaxi® for immunogenic macromolecules (DNA, RNA, Protein) delivery are under development. See for example technologies developed by In-Cell-Art.

In certain aspects, the invention provides nucleic acids comprising sequences encoding envelopes of the invention. In certain embodiments, the nucleic acids are DNAs. In certain aspects, the invention provides expression vectors comprising the nucleic acids of the invention.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding the polypeptide sequence of the sequences of the invention, or a variant thereof or a fragment thereof. Accordingly, in one embodiment, the invention provides an RNA molecule encoding one or more of inventive antibodies. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7mG(5′)ppp(5′)NlmpNp, CleanCap® (e.g., the AG, GG, AU, 3′OMe AG, or 3′OMe G CleanCap®), or ARCA). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. A RNA molecule may have a 3 poly-A tail. It may also include a poly-A polymerase recognition sequence (e.g. AAUIJAAA) near its 3′ end. In some embodiments, a RNA molecule useful with the invention may be single-stranded. In some embodiments, a RNA molecule useful with the invention may comprise synthetic RNA.

The recombinant nucleic acid sequence can be an optimized nucleic acid sequence. Such optimization can increase or alter the immunogenicity of the envelope. Optimization can also improve transcription and/or translation. Optimization can include one or more of the following: low GC content leader sequence to increase transcription; mRNTA stability and codon optimization; addition of a Kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

Methods for in vitro transfection of mRNA and detection of envelope expression are known in the art.

Methods for expression and immunogenicity determination of nucleic acid encoded envelopes are known in the art.

In certain aspects the invention contemplates using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments recombinant proteins are produced in CHO cells.

The immunogenic envelopes can also be administered as a protein prime in combination with a variety of nucleic acid envelope boosts (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated in designs that incorporate appropriate carriers such as peptides for enhancing CD4+ T cell help, known as PADRE, GTH1, GTH2, or any combination thereof. In certain embodiments the compositions are delivered via intramuscular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to, alum, 3M052, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant AS01B but to be less reactogenic using HBsAg as vaccine antigen (Leroux-Roels et al., JABS Conference, April 2013). In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxo1 inhibitor, e.g. 344355 Foxo1 Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody. Non-limiting examples are ipilinuminab and tremelimumab. In certain embodiments, the methods comprise administering a second immunomodulatory agent, wherein the second and first immunomodulatory agents are different.

There are various host mechanisms that control bnAbs. For example, highly somatically mutated antibodies become autoreactive and/or less fit (Immunity 8: 751, 1998; PloS Comp. Biol. 6 e1000800, 2010; J. Thoret. Biol. 164:37, 1993); Polyreactive/autoreactive naïve B cell receptors (unmutated common ancestors of clonal lineages) can lead to deletion of Ab precursors (Nature 373: 252, 1995; PNAS 107: 181, 2010; J. Immunol. 187: 3785, 2011); Abs with long HCDR3 can be limited by tolerance deletion (JI 162: 6060, 1999; JCI 108: 879, 2001). BnAb knock-in mouse models are providing insights into the various mechanisms of tolerance control of MPER BnAb induction (deletion, anergy, receptor editing). Other variations of tolerance control likely will be operative in limiting BnAbs with long HCDR3s, high levels of somatic hypermutations.

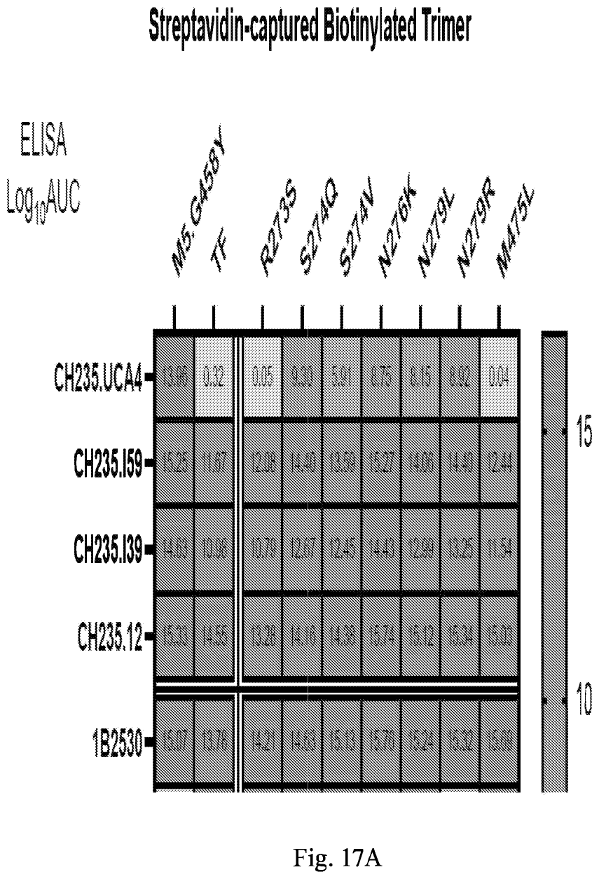

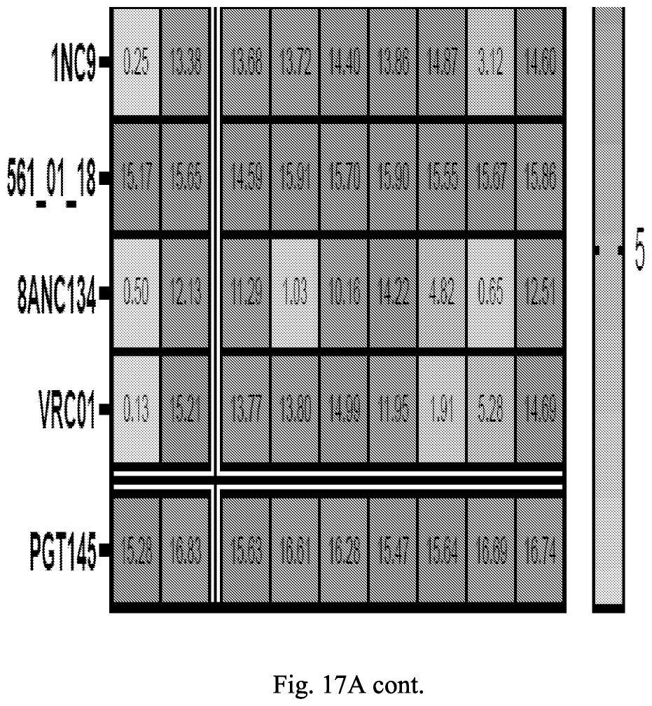

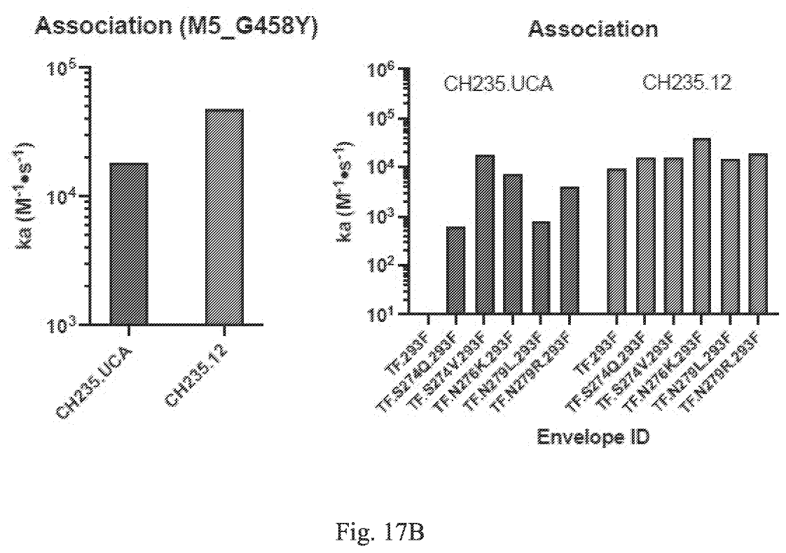

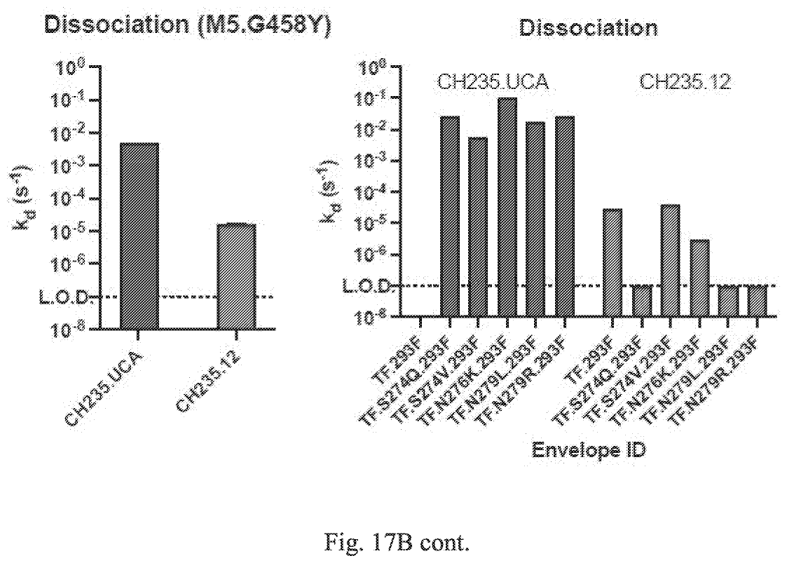

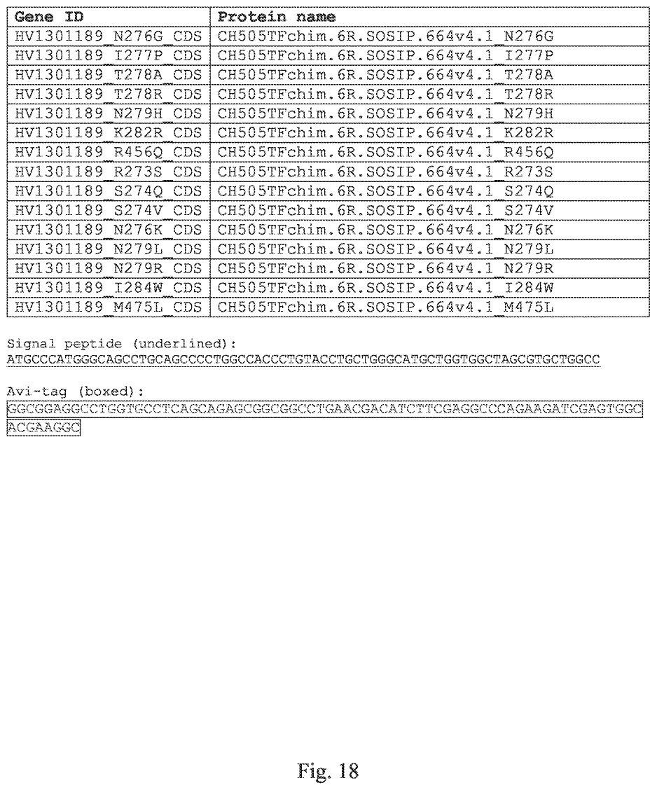

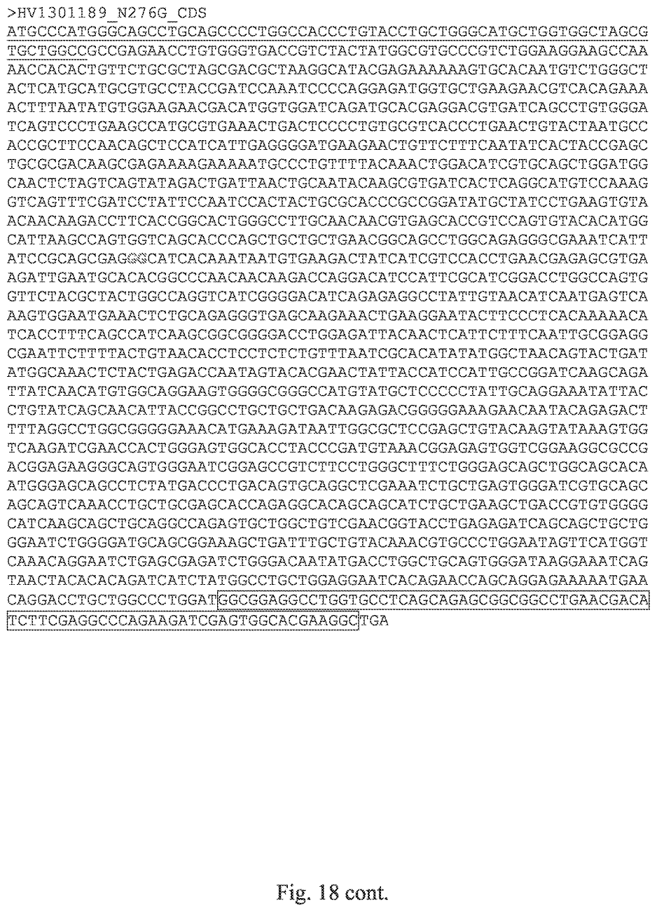

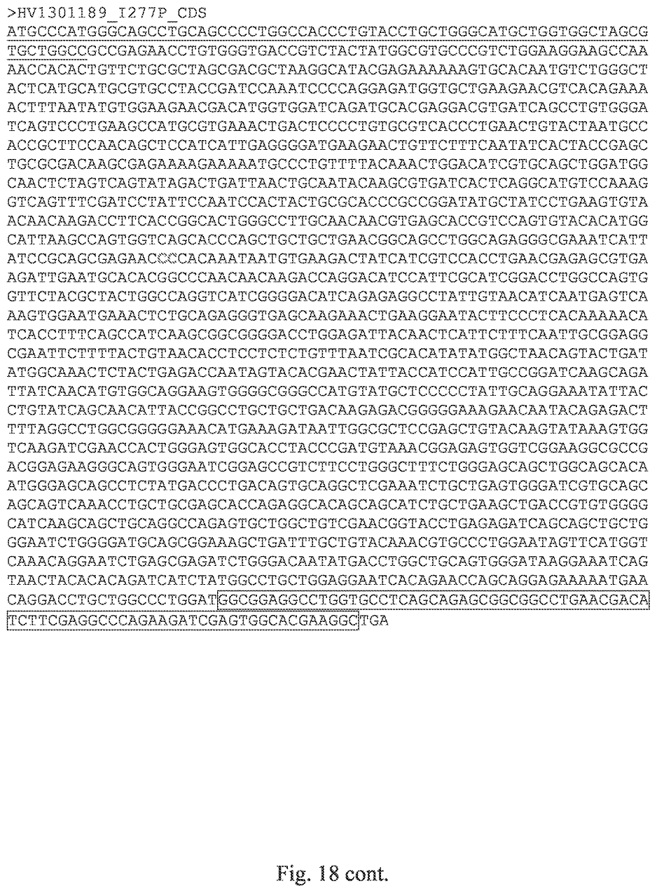

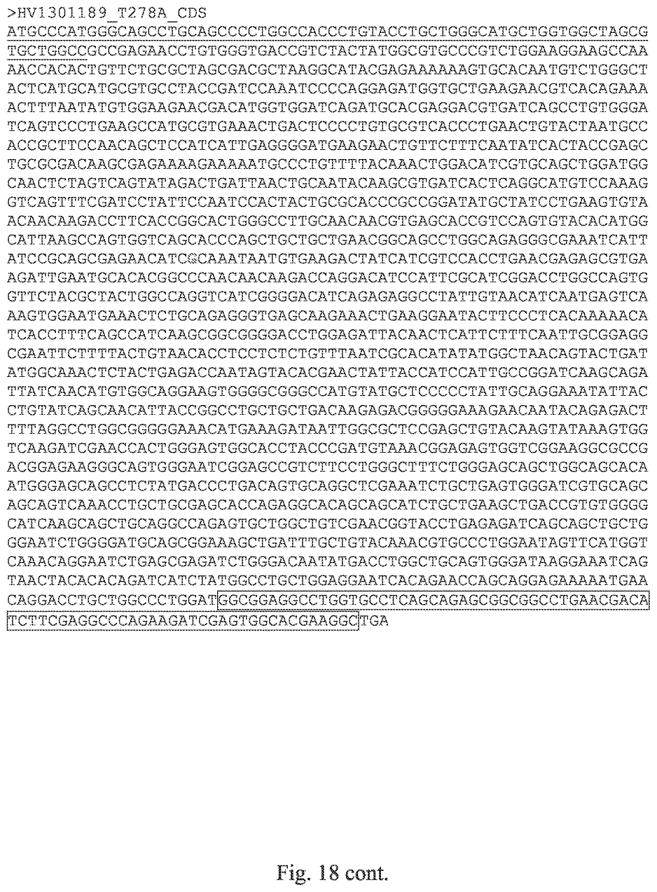

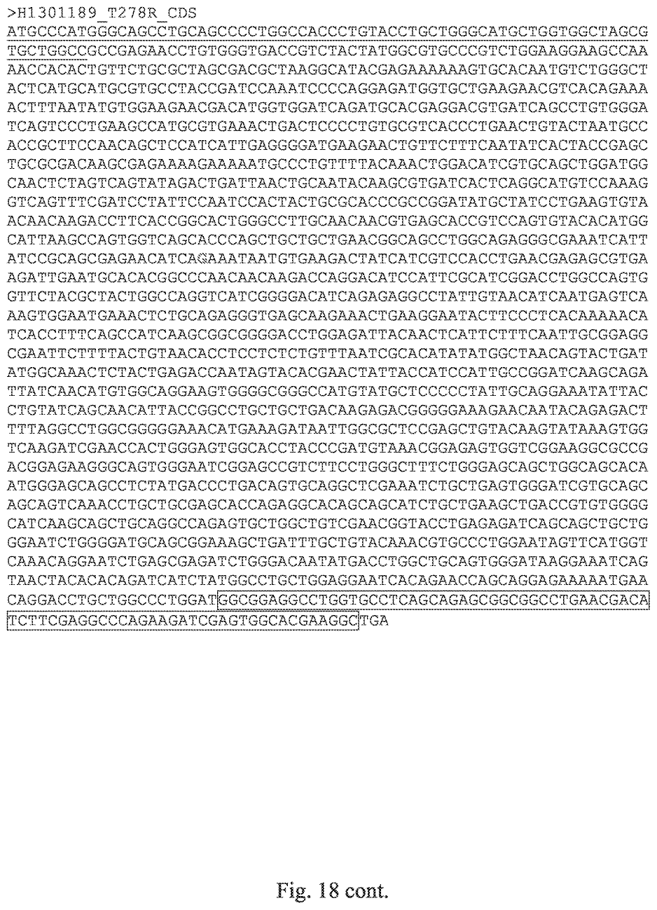

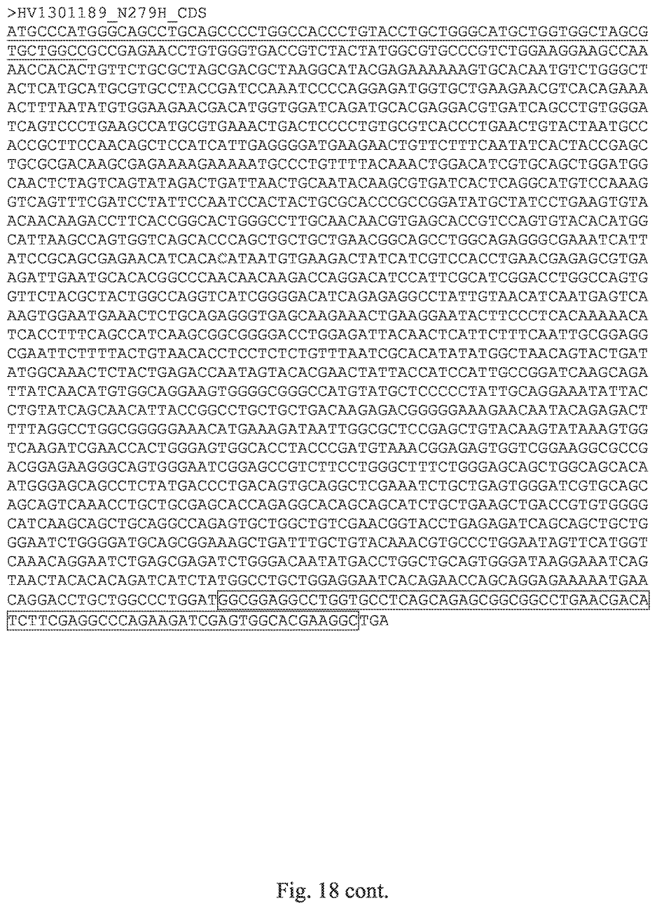

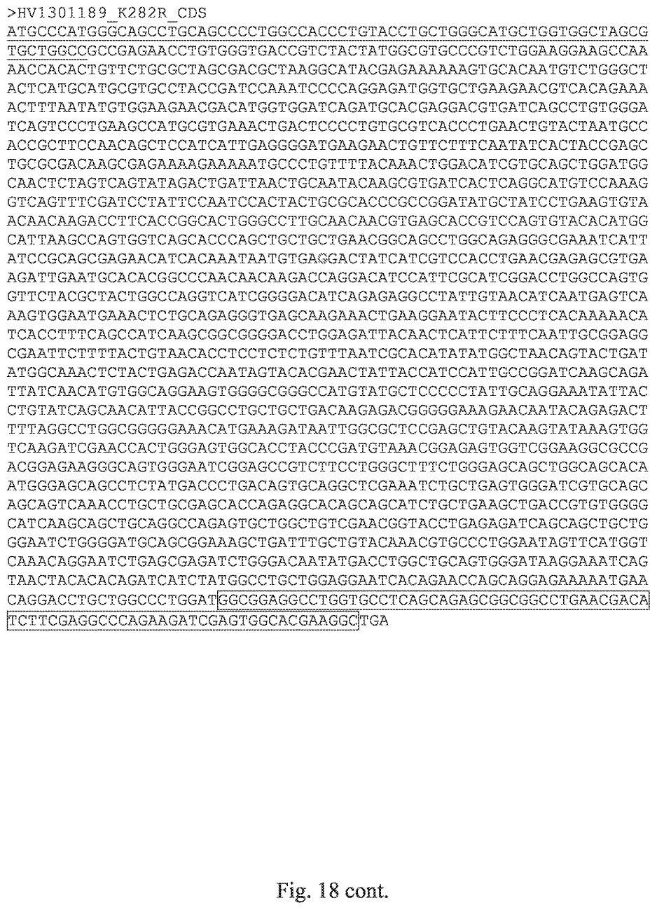

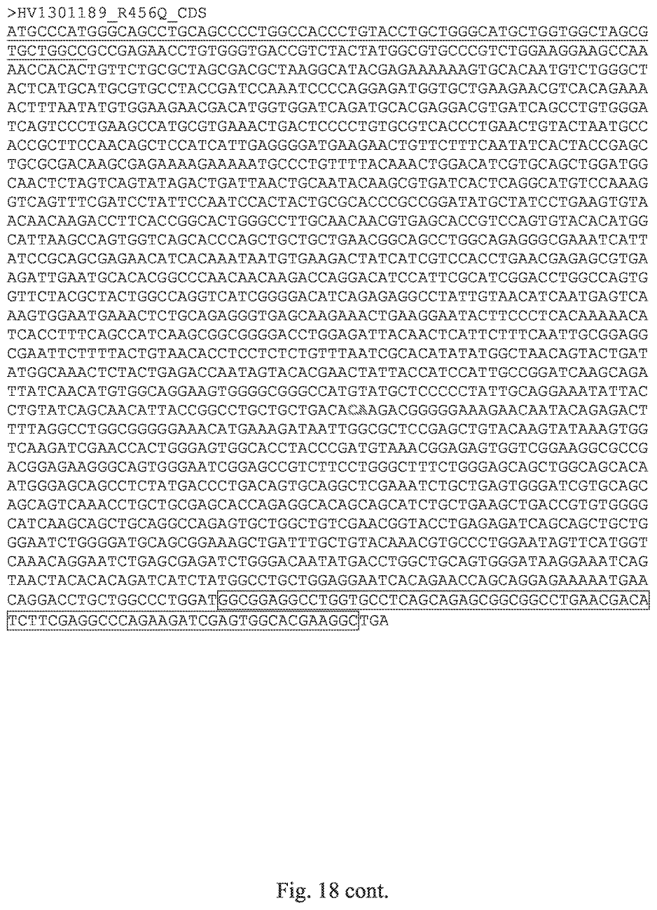

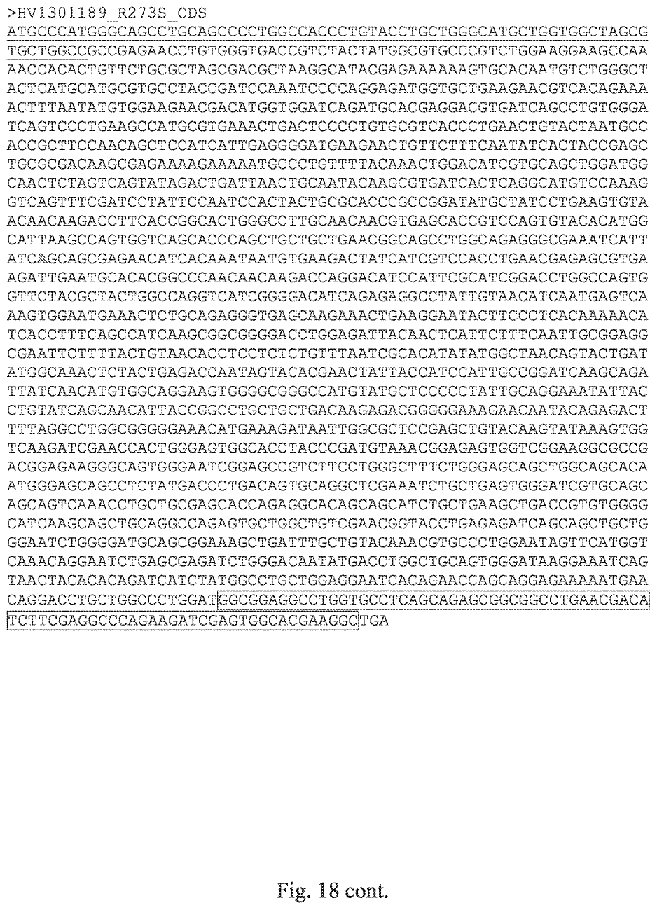

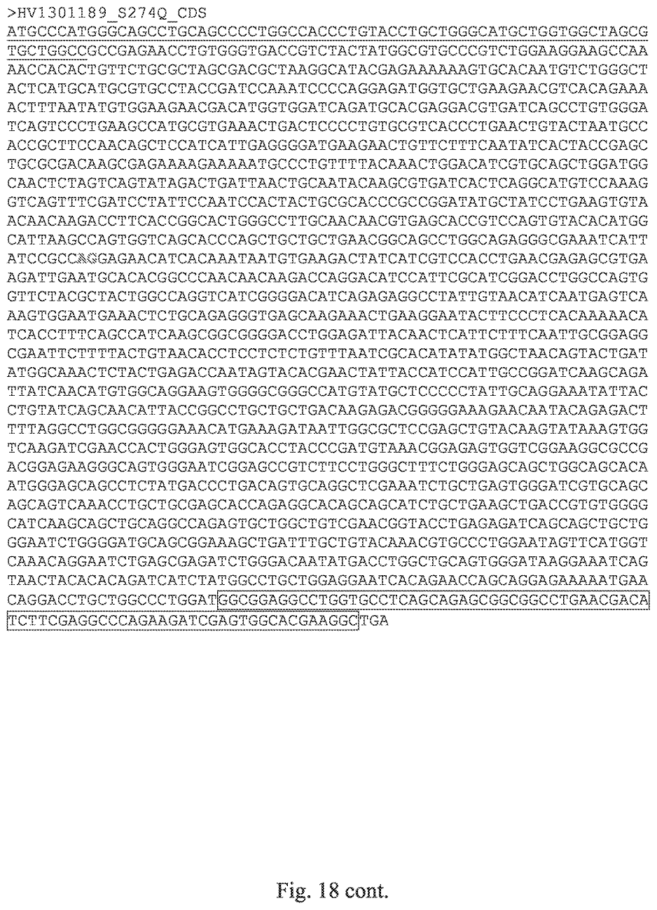

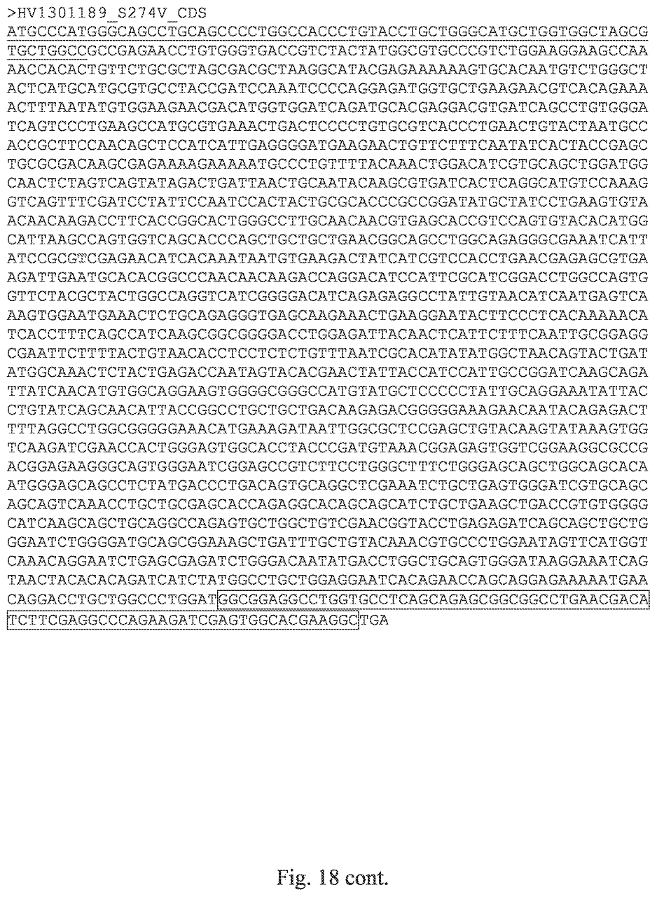

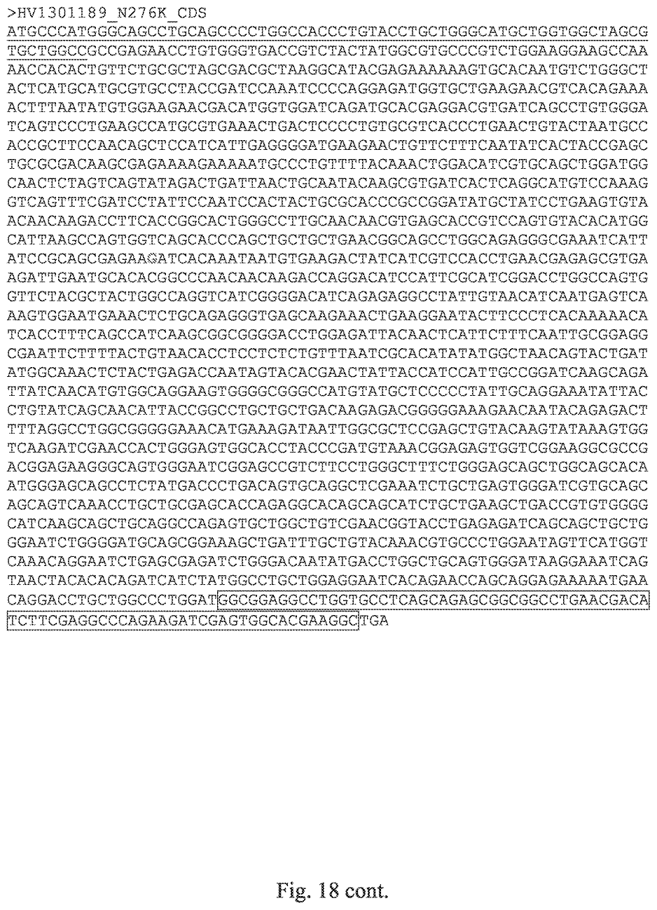

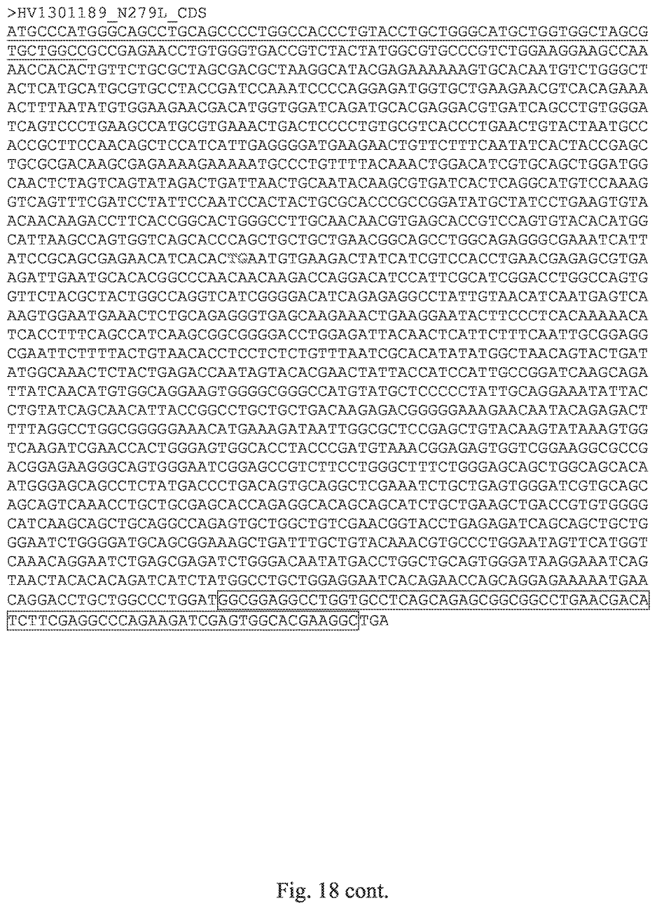

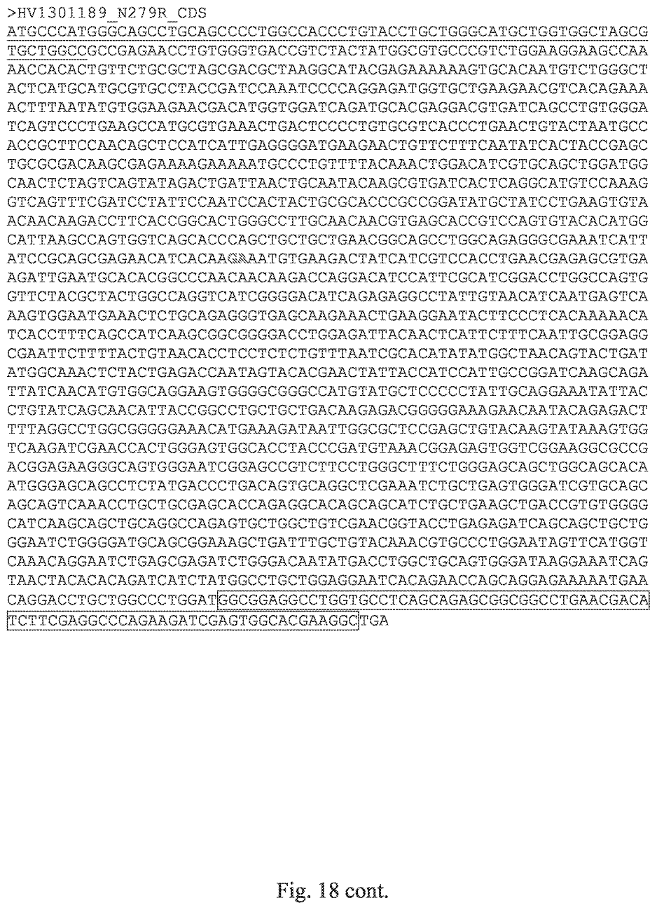

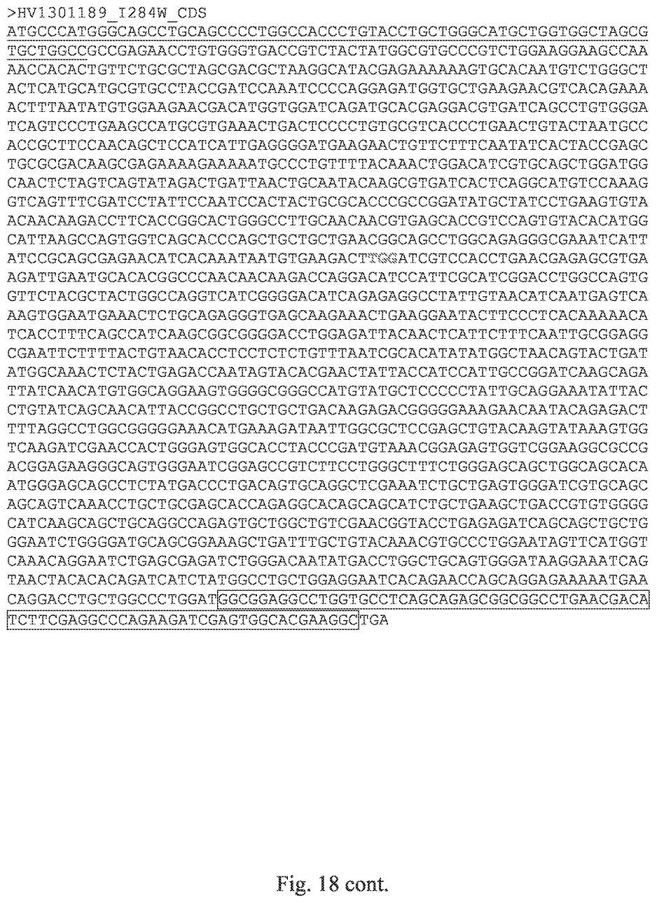

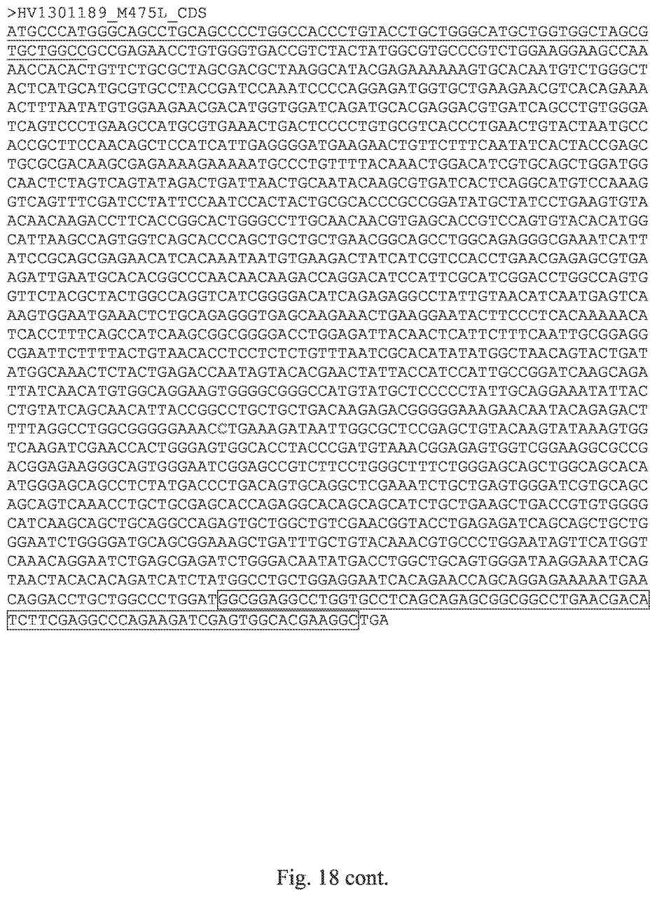

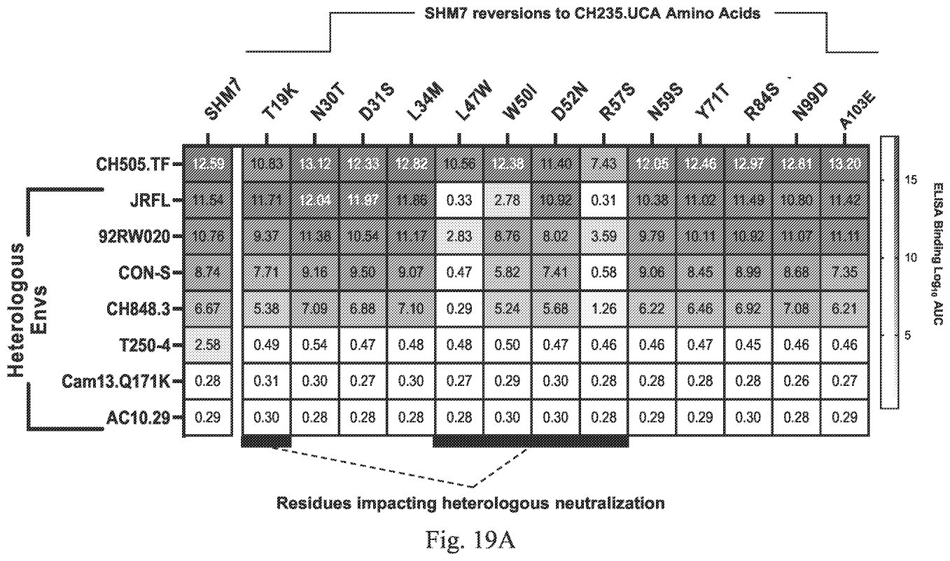

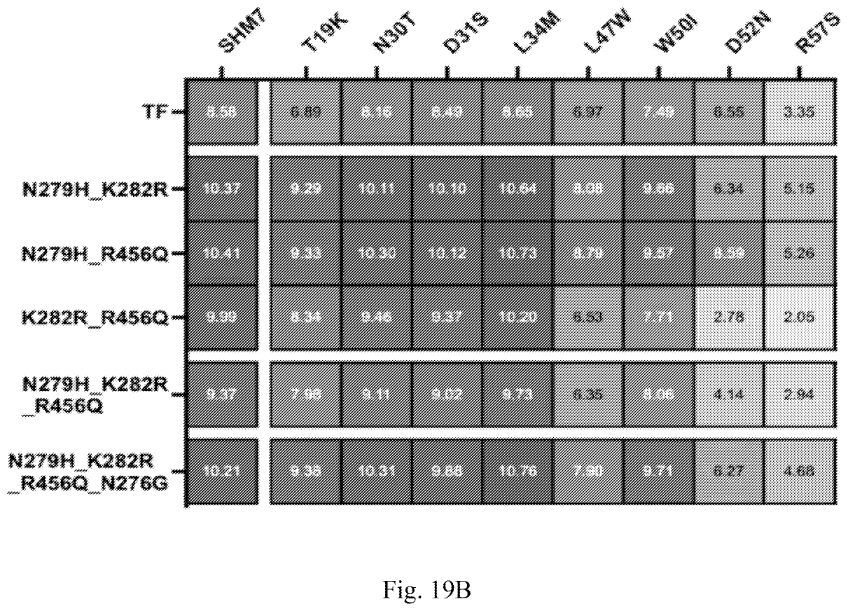

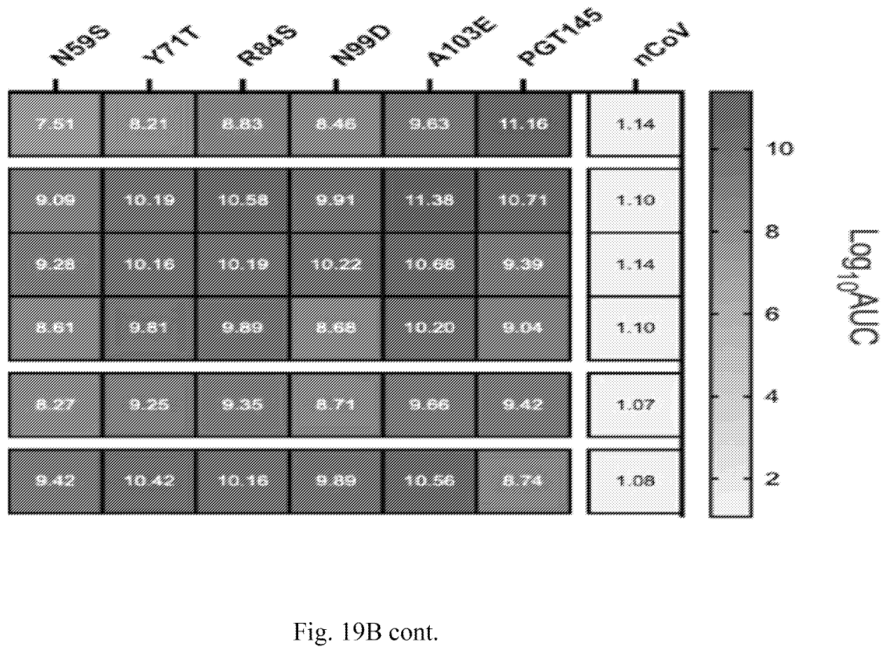

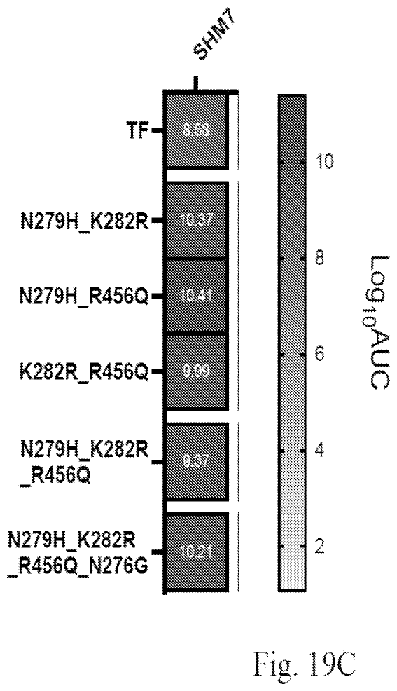

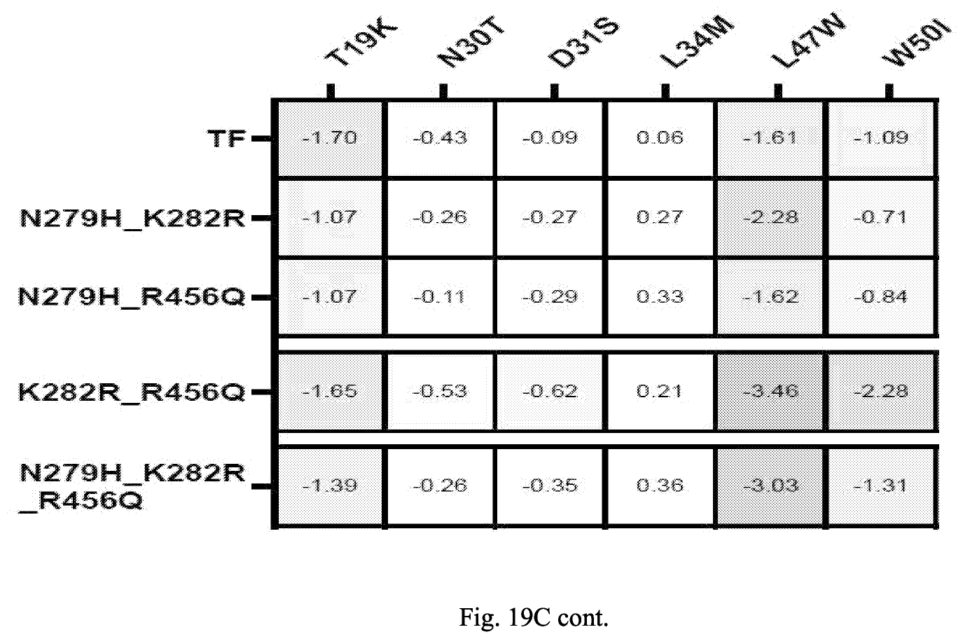

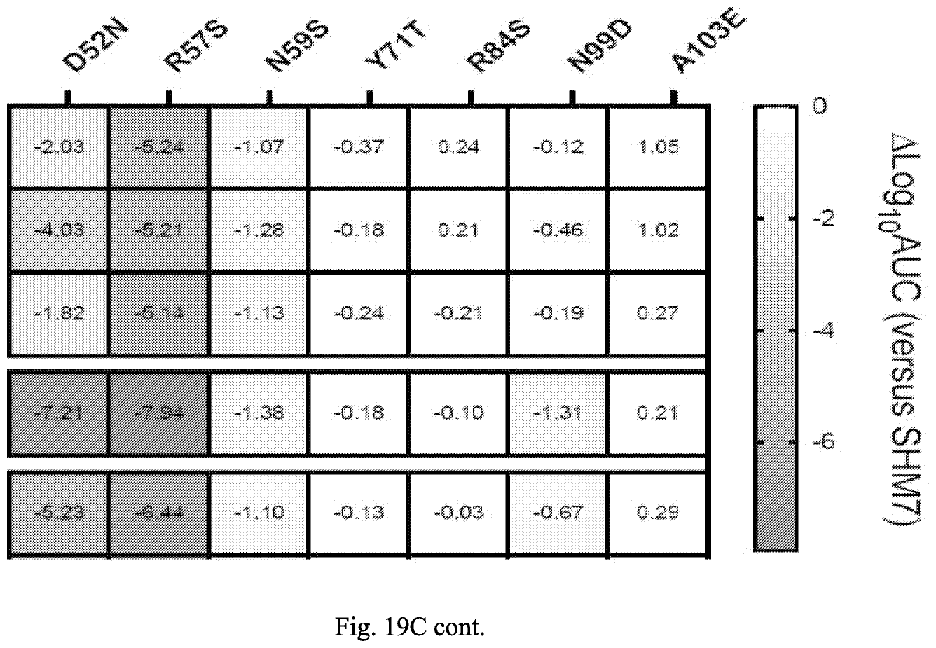

For a summary of CH505 sequences and designs see U.S. Pat. No. 10,968,255, for example, but not limited to, Table 1, FIG. 18-19, and U.S. Pat. No. 10,004,800 (FIG. 17), the contents of each of which are hereby incorporated by reference in their entireties.

It is readily understood that the envelope peptides referenced in various examples and figures comprise a signal peptide/leader sequence. It is well known in the art that HIV-1 envelope peptides is a secretory protein with a signal peptide or leader sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal peptide sequence could be used. In some embodiments the leader sequence is the endogenous leader sequence. Most of the gp120 and gp160 amino acid sequences include the endogenous leader sequence. In other non-limiting examples, the leader sequence is human Tissue Plasminogen Activator (TPA) sequence, human CD5 leader sequence (e.g. MPMiGSLQPLATLYLLGMLVASVLA (SEQ FD NO: 1)). Most of the chimeric designs include CD5 leader sequence. A skilled artisan appreciates that when used as immunogens, and for example when recombinantly produced, the amino acid sequences of these proteins do not comprise the signal peptide/leader sequences.

### HIV-1 Envelope Trimers and Other Envelope Designs

Stabilized HIV-1 Env trimer immunogens show enhanced antigenicity for broadly neutralizing antibodies and are not recognized by non-neutralizing antibodies. Envelope modifications and designs include, but are not limited to, trimers that are further multimerized, and/or used as particulate, high-density array in liposomes or other particles, for example but not limited to nanoparticles. Any one of the envelopes of the invention could be designed and expressed as described herein.

A stabilized chimeric SOSIP designs can be used to generate CH505 trimers. This design is applicable to diverse viruses from multiple clades. SOSIP designs can be applied to the envelopes disclosed herein including those in FIGS. 1 and 18.

Elicitation of neutralizing antibodies is one goal for antibody-based vaccines. Neutralizing antibodies target the native trimeric HIV-1 Env on the surface virions. The trimeric HIV-1 envelope protein consists of three protomers each containing a gp120 and gp41 heterodimer. Recent immunogen design efforts have generated soluble near-native mimics of the Env trimer that bind to neutralizing antibodies but not non-neutralizing antibodies. The recapitulation of the native trimer could be a key component of vaccine induction of neutralizing antibodies. Neutralizing Abs target the native trimeric HIV-1 Env on the surface of viruses (Poignard et al. J Virol. 2003 January; 77(1):353-65; Parren et al. J Virol. 1998 December; 72(12):10270-4.; Yang et al, J Virol. 2006 November; 80(22):11404-8.). The HIV-1 Env protein consists of three protomers of gp120 and gp4I heterodimers that are noncovalently linked together (Center et al J Virol. 2002 August; 76(15):7863-7.). Soluble near-native trimers preferentially bind neutralizing antibodies as opposed to non-neutralizing antibodies (Sanders et al PLoS Pathog. 2013 September; 9(9): e1003618).

Provided are engineered trimeric immunogens derived from multiple viruses from CH505. We generated chimeric 6R.SOSIP.664, chimeric disulfide stabilized (DS) 6R.SOSIP.664 (Kwon et al Nat Struct Mol Biol. 2015 July; 22(7): 522-531.), chimeric 6R.SOSIP.664v4.1 (DeTaeye et al. Cell. 2015 Dec. 17; 163(7):1702-15. doi: 10.1016/j.cell.2015.11.056), and chimeric 6R.SOSIP.664v4.2 (DeTaeye et al. Cell. 2015 December 17; 163(7):1702-15. doi: 10.1016/j.cell.2015.l 1.056). The 6R.SOSIP.664 is the basis for all of these designs and is made as a chimera of C.CH0505 and A.BG505. The gp120 of C.CH505 was fused with the BG505 inner domain gp120 sequence within the alpha helix 5 (α5) to result in the chimeric protein. The chimeric gp120 is disulfide linked to the A.BG505 gp4l as outlined by Sanders et al. (PLoS Pathog. 2013 September; 9(9): e1003618). These immunogens were designed as chimeric proteins that possess the BG505 gp41 connected to the CH505 gp120, since the BG505 strain is particularly adept at forming well-folded, closed trimers. This envelope design retains the CH505 CD4 binding site that is targeted by the CH103 and CH235 broadly neutralizing antibody lineages that were isolated from CH505.

Based on the various designs, any other suitable envelope, for example but not limited to CH505 envelopes as described in U.S. Pat. No. 10,004,800, incorporated herein by reference, can be designed to include one or more of the mutations described herein (e.g., mutation of one or more of amino acid residue R273, S274, N276, N279, I277, T278, K282, 1284, R456, or M475 (HXB2 numbering), including, but not limited to, N276C, I277P, T278A, T278R, K282R, R456Q, R273S, S274Q S274V, N276K, N279H, N279L, N279R, 1284W, or M475L). In some embodiments, the envelope sequences further comprise SOSIP designs.

Recombinant envelopes as trimers could be produced and purified by any suitable method. For a non-limiting example of purification methods see Ringe R1P, Yasmeen A, Ozorowski G, Go E P, Pritchard L K, Guttman M, Ketas T A, Cottrell C A, Wilson I A, Sanders R W, Cupo A, Crispin M, Lee K K, Desaire H, Ward A B, Klasse P J, Moore J P. 2015. Influences on the design and purification of soluble, recombinant native-like HIV-1 envelope glycoprotein trimers. J Virol 89:12189-12210. doi:10.1128/JVI.01768-15.

### Multimeric Envelopes

Presentation of antigens as particulates reduces the B cell receptor affinity necessary for signal transduction and expansion (see Baptista et al. EMBO J. 2000 Feb. 15; 19(4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See e.g. He et al. Nature Communications 7, Article number: 12041 (2016), doi:10,1038/ncomms12041; Bamrungsap et al. Nanomedicine, 2012, 7 (8), 1253-12⁷1.

Multimeric nanoparticles that comprise and/or display HIV envelope protein or fragments on their surface can be used a vaccine immunogens.

In some instances, the nucleic acid encoding an antigen (e.g., an HIV-envelope polypeptide) is fused via a linker/spacer to a nucleic acids sequence encoding a protein which can self-assemble. Upon translation, a fusion protein is made that can self-assemble into a multimeric complex—also referred to as a nanoparticle displaying multiple copies of the antigen. In other instances, the protein antigen could be conjugated to the self-assembling protein via an enzymatic reaction, thereby forming a nanoparticle displaying multiple copies of the antigen. Non-limiting embodiments of enzymatic conjugation include without limitation sortase mediated conjugation. In some embodiments, linkers for use in any of the designs of the invention could be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine.

Ferritin is a well-known protein that self-assembles into a hollow particle composed of repeating subunits. In some species ferritin nanoparticles are composed of 24 copies of a single subunit, whereas in other species it is composed of 12 copies each of two subunits.

To improve the interaction between the naïve B cell receptor and immunogens, in some embodiments, an envelope design is created so the envelope is presented on particles, e.g. but not limited to nanoparticle. In some embodiments, the HIV-1 envelope trimer could be fused to ferritin. Ferritin protein self assembles into a small nanoparticle with three-fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g., Sliepen et al. Retrovirology201512:82, DOI: 10.1186/s12977-015-0210-4.

Any suitable ferritin sequence could be used. In non-limiting embodiments, ferritin sequences are disclosed in U.S. Pat. No. 10,961,283, incorporated herein by reference.

Ferritin nanoparticle linkers: The ability to form HIV-1 envelope ferritin nanoparticles relies self-assembly of 24 ferritin subunits into a single ferritin nanoparticle. The addition of a ferritin subunit to the C-terminus of HIV-1 envelope may interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope could be sterically hindering the association of ferritin subunits. Thus, we designed ferritin with elongated glycine-serine linkers to further distance the envelope from the ferritin subunit. To make sure that the glycine linker is attached to ferritin at the correct position, we created constructs that attach at second amino acid position or the fifth amino acid position. The first four N-terminal amino acids of natural Helicobacter pylori ferritin are not needed for nanoparticle formation but may be critical for proper folding and oligomerization when appended to envelope. Thus, constructs can be designed constructs with and without the leucine, serine, and lysine amino acids following the glycine-serine linker. The goal will be to find a linker length that is suitable for formation of envelope nanoparticles when ferritin is appended to most envelopes. Any suitable linker between the envelope and ferritin could be used, so long as the fusion protein is expressed and the trimer is formed.

The nanoparticle immunogens are composed of various forms of HIV-1 envelope protein, e.g. without limitation envelope trimer, and self-assembling protein, e.g. without limitation ferritin protein. Any suitable ferritin could be used in the immunogens of the invention. In non-limiting embodiments, the ferritin is derived from Helicobacter pylori. In non-limiting embodiments, the ferritin is insect ferritin. In non-limiting embodiments, each nanoparticle displays 24 copies of the envelope protein on its surface.

Another approach to multimerize expression constructs uses staphylococcus Sortase A transpeptidase ligation to conjugate inventive envelope trimers, for e.g. but not limited to cholesterol. The trimers can then be embedded into liposomes via the conjugated cholesterol. To conjugate the trimer to cholesterol either a C-terminal LPXTG tag (SEQ ID NO: 8) or a N-terminal pentaglycine repeat tag (SEQ ID NO: 9) is added to the envelope trimer gene. Cholesterol is also synthesized with these two tags. In a non-limiting embodiment, a C-terminal tag is LPXTGG, where X signifies any amino acid but most commonly Ala, Ser, Glu (SEQ ID NO: 46). Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles. Any suitable ferritin can be used.

The invention provides design of envelopes and trimer designs wherein the envelope comprises a linker which permits addition of a lipid, such as but not limited to cholesterol, via a Sortase A reaction. See e.g., Tsukijji, S. and Nagamune, T. (2009), Sortase-Mediated Ligation: A Gift from Gram-Positive Bacteria to Protein Engineering. ChemBioChem, 10: 787-798. DOI: 10.1002chic.200800724; Proft T. Sortase-mediated protein ligation an emerging biotechnology tool for protein modification and 26mmobilization. Biotechnol Lett (2010) 32: 1. Doi: 10.100Z1/s10529-009-0116-0; Lena Schmohi, Dirk Schwarzer, Sortase-mediated ligations for the site-specific modification of proteins, Current Opinion in Chemical Biology, Volume 22, October 2014, Pages 122-128, ISSN 1367-5931 dx.doi.org/10.1016j.cbpa.2014.09.020: ITabata et al. Anticancer Res. 2015 August; 35(8)g411-7: Pritz et al. J. Org. Chem. 2007, 72, 3909-3912.

The lipid modified envelopes and tri-ers could be formulated as liposomes. Any suitable liposome composition is contemplated.

The lipid modified and multimerized envelopes and trimers could be formulated as liposomes. Any suitable liposome composition is contemplated.

Table 1 shows a summary of sequences described herein.

| Protein ID | Protein name | SEQ ID NO: |
|---|---|---|
| HV1301189_{—} | CH505TFchim.6R.SOSIP.664v4.1_N276G | 10 (amino acid) - FIG. 1 |
| N276G | | 31 (nucleic acid) - FIG. 18 |
| HV1301189_{—} | CH505TFchim.6R.SOSIP.664v4.1_I277P | 11 (amino acid) - FIG. 1 |
| I277P | | 32 (nucleic acid) - FIG. 18 |
| HV1301189_{—} | CH505TFchim.6R.SOSIP.664v4.1_T278A | 12 (amino acid) - FIG. 1 |
| T278A | | 33 (nucleic acid) - FIG. 18 |
| HV1301189_{—} | CH505TFchim.6R.SOSIP.664v4.1_T278R | 13 (amino acid) - FIG. 1 |
| T278R | | 34 (nucleic acid) - FIG. 18 |
| HV1301189_{—} | CH505TFchim.6R.SOSIP.664v4.1_K282R | 15 (amino acid) - FIG. 1 |
| K282R | | 36 (nucleic acid) - FIG. 18 |
| HV1301189_{—} | CH505TFchim.6R.SOSIP.664v4.1_R456Q | 16 (amino acid) - FIG. 1 |
| R456Q | | 37 (nucleic acid) - FIG. 18 |
| HV1301189_{—} | CH505TFchim.6R.SOSIP.664v4.1_R273S | 17 (amino acid) - FIG. 1 |
| R273S | | 38 (nucleic acid) - FIG. 18 |
| HV1301189_{—} | CH505TFchim.6R.SOSIP.664v4.1_S274Q | 18 (amino acid) - FIG. 1 |
| S274Q | | 39 (nucleic acid) - FIG. 18 |
| HV1301189_{—} | CH505TFchim.6R.SOSIP.664v4.1_S274V | 19 (amino acid) - FIG. 1 |
| S274V | | 40 (nucleic acid) - FIG. 18 |
| HV1301189_{—} | CH505TFchim.6R.SOSIP.664v4.1_N276K | 20 (amino acid) - FIG. 1 |
| N276K | | 41 (nucleic acid) - FIG. 18 |
| HV1301189_{—} | CH505TFchim.6R.SOSIP.664v4.1_N279L | 21 (amino acid) - FIG. 1 |
| N279L | | 42 (nucleic acid) - FIG. 18 |
| HV1301189_{—} | CH505TFchim.6R.SOSIP.664v4.1_N279R | 22 (amino acid) - FIG. 1 |
| N279R | | 43 (nucleic acid) - FIG. 18 |
| HV1301189_{—} | CH505TFchim.6R.SOSIP.664v4.1_I284W | 23 (amino acid) - FIG. 1 |
| I284W | | 44 (nucleic acid) - FIG. 18 |
| HV1301189_{—} | CH505TFchim.6R.SOSIP.664v4.1_M475L | 24 (amino acid) - FIG. 1 |
| M475L | | 45 (nucleic acid) - FIG. 18 |
| HV1301189_{—} | CH505TFchim.6R.SOSIP.664v4.1_N279H | 14 (amino acid) - FIG. 1 |
| N279H_CDS | | 35 (nucleic acid) - FIG. 18 |

The trimer could be incorporated in a nanoparticle, including without limitation any ferritin based nanoparticle.

Throughout the application amino acid positions numbers refer to HXB2 numbering.

Any of the immunogens herein may be encoded by a nucleic acid. It will be understood that non-identical nucleic acid sequences may encode the same amino acid sequence. As such these examples do not exclude nucleic acid sequences that encode immunogens with the same amino acid sequence but possess different nucleic acid sequences.

### Exemplary Embodiments

In certain aspects, the invention provides a recombinant HIV-1 envelope comprising all the consecutive amino acids between the signal peptide and avi-tag of SEQ ID NOs:10-24. In some embodiments, the recombinant HIV-1 envelope comprises all the consecutive amino acids between the signal peptide and avi-tag of SEQ ID NO: 14. In some embodiments, the recombinant HIV-1 envelope comprising all the consecutive amino acids between the signal peptide and avi-tag of SEQ ID NO: 14 further comprises mutation K282R. In some embodiments, the recombinant HIV-1 envelope comprising all the consecutive amino acids between the signal peptide and avi-tag of SEQ ID NO: 14 further comprises mutation R456Q. In some embodiments, the recombinant HIV-1 envelope comprising all the consecutive amino acids between the signal peptide and avi-tag of SEQ ID NO: 14 further comprises mutation K282R and R456Q. In some embodiments, the recombinant HIV-1 envelope comprises all the consecutive amino acids between the signal peptide and avi-tag of SEQ ID NO: 15. In some embodiments, the recombinant HIV-1 envelope comprising all the consecutive amino acids between the signal peptide and avi-tag of SEQ ID NO: 15 further comprises mutation R456Q. In some embodiments, the recombinant HIV-1 envelope comprises all the consecutive amino acids between the signal peptide and avi-tag of SEQ ID NO: 16. In some embodiments, the recombinant HIV-1 envelope comprises all the consecutive amino acids between the signal peptide and avi-tag of SEQ ID NO: 10. In some embodiments, any of the recombinant HIV-1 envelopes further comprises mutation N276G. In some embodiments, the recombinant HIV-1 envelope further comprises the amino acid sequence of the avi-tag (e.g., in certain aspects, the invention provides a recombinant HIV-1 envelope comprising all the consecutive amino acids after the signal peptide of SEQ ID NOs:10-24). In some embodiments, the envelope is a protomer comprised in a trimer.

In certain aspects, the invention provides a nucleic acid encoding any one of the recombinant HIV-1 envelopes described herein. In some embodiments, the nucleic acid comprises the portion of SEQ ID NOs: 31-45 encoding all the consecutive amino acids between the signal peptide and avi-tag in some embodiments, the nucleic acid sequence further comprises a portion encoding a signal peptide. In some embodiments, the nucleic acid sequence further comprises a portion encoding an avi-tag.

In certain aspects, the invention provides a recombinant trimer comprising three identical protomers of an HIV-1 envelope comprising all the consecutive amino acids between the signal peptide and avi-tag of SEQ ID NOs:10-24. In some embodiments, the HIV-1 envelope comprises all the consecutive amino acids between the signal peptide and avi-tag of SEQ ID NO: 14. In some embodiments, the HIV-1 envelope comprising all the consecutive amino acids between the signal peptide and avi-tag of SEQ ID NO: 14 further comprises mutation K282R. In some embodiments, the HIV-1 envelope comprising all the consecutive amino acids between the signal peptide and avi-tag of SEQ ID NO: 14 further comprises mutation R456Q. In some embodiments, the HIV-1 envelope comprising all the consecutive amino acids between the signal peptide and avi-tag of SEQ ID NO: 14 further comprises mutation K282R and R456Q. In some embodiments, the HIV-1 envelope comprises all the consecutive amino acids between the signal peptide and avi-tag of SEQ ID NO: 15. In some embodiments, the HIV-1 envelope comprising all the consecutive amino acids between the signal peptide and avi-tag of SEQ ID NO: 15 further comprises mutation R456Q. In some embodiments, the HIV-1 envelope comprises all the consecutive amino acids between the signal peptide and avi-tag of SEQ ID NO: 16. In some embodiments, the HIV-1 envelope comprises all the consecutive amino acids between the signal peptide and avi-tag of SEQ ID NO: 10. In some embodiments, any of the HIV-1 envelopes further comprises mutation N276G.

In certain aspects, the invention provides an immunogenic composition comprising the recombinant trimer described herein and a carrier. In certain aspects, the invention provides an immunogenic composition comprising the nucleic acid described herein and a carrier. In certain aspects, the invention provides an immunogenic composition comprising the recombinant HIV-1 envelopes described herein and a carrier. In some embodiments, the immunogenic composition further comprises an adjuvant. In some embodiments, the nucleic acid is operably linked to a promoter, and optionally the nucleic acid is inserted in an expression vector.

In certain aspects, the invention provides a method of inducing an immune response in a subject comprising administering a composition comprising any suitable form of a nucleic acid(s) described herein or the envelope described herein in an amount sufficient to induce an immune response. In some embodiments, the nucleic acid encodes a protomer of a SOSIP trimer. In some embodiments, the envelope is a protomer of a SOSIP trimer. In some embodiments, the composition further comprises an adjuvant. In some embodiments, the method further comprises administering an agent which modulates host immune tolerance. In some embodiments, the envelope administered is multimerized in a liposome or nanoparticle. In some embodiments, the method further comprises administering one or more additional HIV-1 immunogens to induce a T cell response.

In certain aspects, the invention provides a composition comprising a nanoparticle and a carrier, wherein the nanoparticle comprises any one of the envelopes described herein. In some embodiments, the nanoparticle is ferritin self-assembling nanoparticle. In some embodiments, the composition is an immunogenic composition.

In certain aspects, the invention provides a composition comprising a nanoparticle and a carrier, wherein the nanoparticle comprises any one of the trimers described herein. In some embodiments, the nanoparticle is ferritin self-assembling nanoparticle. In some embodiments, the nanoparticle comprises multimers of trimers. In some embodiments, the composition is an immunogenic composition.

In certain aspects, the invention provides a composition comprising a nanoparticle and a carrier, wherein the nanoparticle comprises any one of the nucleic acids described herein. In some embodiments, the nanoparticle is a lipid nanoparticle. In some embodiments, the composition is administered as a prime. In some embodiments, the composition is administered as a boost. In some embodiments, the composition is an immunogenic composition.

In certain aspects, the invention provides a method of inducing an immune response in a subject comprising administering an immunogenic composition described herein in an amount sufficient to induce an immune response.

In certain aspects, the invention provides a composition comprising the recombinant trimer described herein and a carrier. In certain aspects, the invention provides a composition comprising the nucleic acid described herein and a carrier. In certain aspects, the invention provides a composition comprising the envelope described herein and a carrier.

### EXAMPLES

### Example 1—Evolving C D4 Binding Site Immunogens Via Antibody Lineage Selection and Mammalian Cell Display

### Background

Anti-HIV broadly neutralizing antibodies (bnAbs) typically develop through extensive co-evolution with virus during chronic infection. Sequential vaccination, targeting these potent humoral responses, assumes an optimized sequence of differing envelopes can similarly mature B cell receptors toward neutralization breadth and potency. Previous work demonstrates a priming immunogen (CH505TF.N279K.G458Y/GnTI-), based on sequences from the CH505 viral quasispecies, readily induces potent, autologous CD4 binding site (CD4bs) antibodies reminiscent of the CH235.12 bnAb class. Further optimization of this immunogen would aim to engage additional CD4 mimicking bnAb lineages, including the highly similar VH1-46 bnAb class lineages 1-18 and 8ANC131. The next steps for generating a complete vaccine are to develop immunogens which select for early/mid stage intermediate antibodies that have accumulated improbable mutations of interest. Here, we utilized directed evolution to isolate mutants of CH505TF which can serve as priming immunogens or boosting immunogens with high affinity for an early intermediate antibody with heterologous neutralization

### Methods

Single mutant envelope sequences were randomly generated throughout the CD4bs epitope (D, V5, and CD4 binding loops) and expressed on the surface of 293T cells. Antibody-reactive envelopes were sorted and enriched using fluorescence-activated cell sorting. Cell display cultures with enhanced antibody reactivity were sequenced via Illumina short read technology and highly abundant envelope species expressed via transient transfection. Antibody neutralization, reactivity and kinetics were assessed via TZM-bI pseudotyped virus assays, ELISAs, and bio-layer interferometry, respectively.

### Results

An optimized CH235 intermediate (named CH235.SHM7) was created by adding 13 improbable, class-conserved, or structurally crucial lineage mutations to UCA. This antibody achieves potent autologous and early heterologous neutralization with fewer mutations, limited exclusively to the heavy chain, versus a comparable CH235 intermediate. CH235 SHM 7 library sorting yielded seven stable single mutants, four targeting the 276 glycan sequon, as well as N279H, K282R, and R456Q with evidence of improved kinetics (down to picomolar affinity for CH235.12) and binding for CH235/8ANC131 lineage members. In early, lower stringency (higher antibody concentration) sorting K282R dominated, while later, higher stringency pointed toward glycan sequon dominance. In contrast, sorting the single mutant library with UCA yielded 8 highly reactive mutations with 7 originating in the D loop (R273S, S274Q, S274V, N276K, N279L, N279K, and 1284W), and one more within the V5 loop (M475L).

### CONCLUSIONS

Directed evolution of CH505-derived envelope sequences using both an optimized early intermediate and the UCA shows loop D as an immunological barrier, with numerous single mutant candidates that readily improve binding. Additionally, multiple priming candidates have been identified that could enhance current immunogens that include the N279K and G458Y mutations or substitute for one or both of these mutations.

### Example 2: CH505.TF Immunogen Design

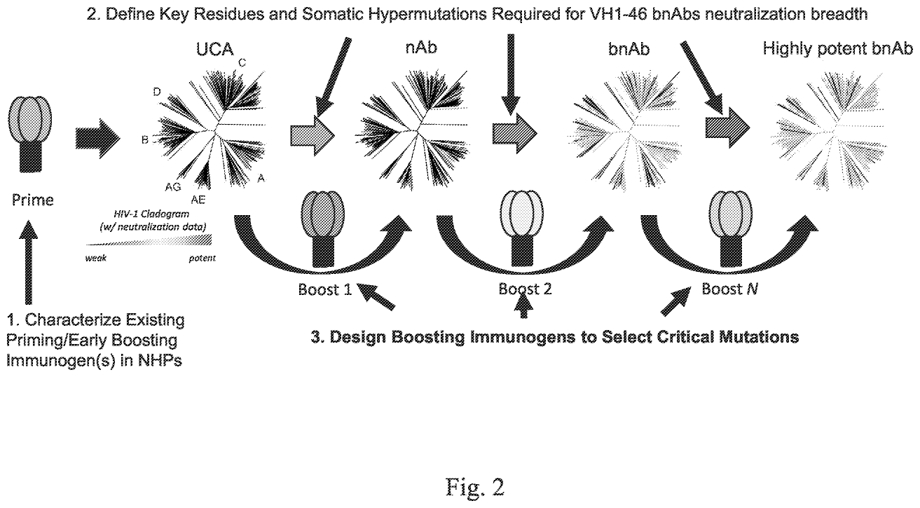

FIG. 2-11 disclose CH505.TF immunogen design.

To design CH505.TF immunogens, the existing priming/early boosting immunogen(s) in nonhuman primates (NHPs) were first characterized (FIG. 2). Key residues and somatic hypermutations required for VH1-46 bnAbs neutralization breadth were defined. Then, boosting immunogens were designed to select critical mutations.

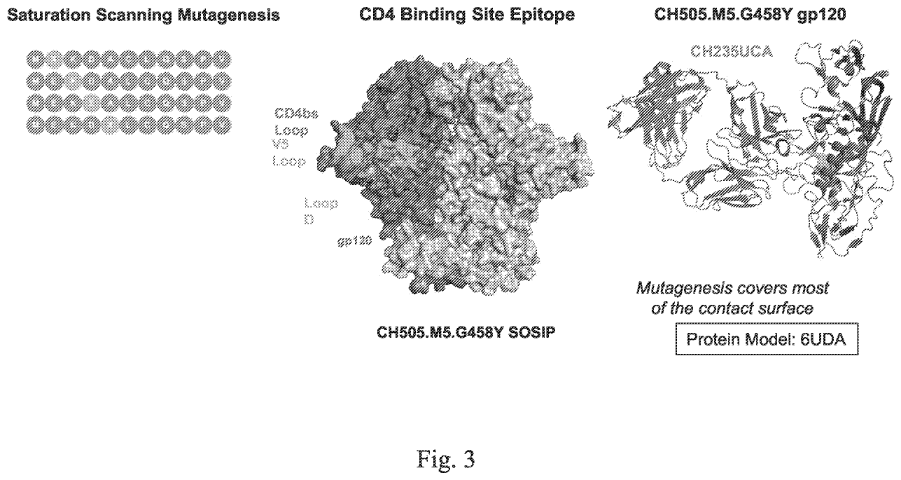

To understand whether SHM7 can select for a higher affinity immunogen, a mutagenesis library was constructed (FIG. 3). Saturation scanning mutagenesis was performed by replacing each target residues with all other possible amino acids, as shown in FIG. 3. It showed that the mutagenesis covered most of the contact surface between CH235UCA and CH505.M5.G458Y gp120.

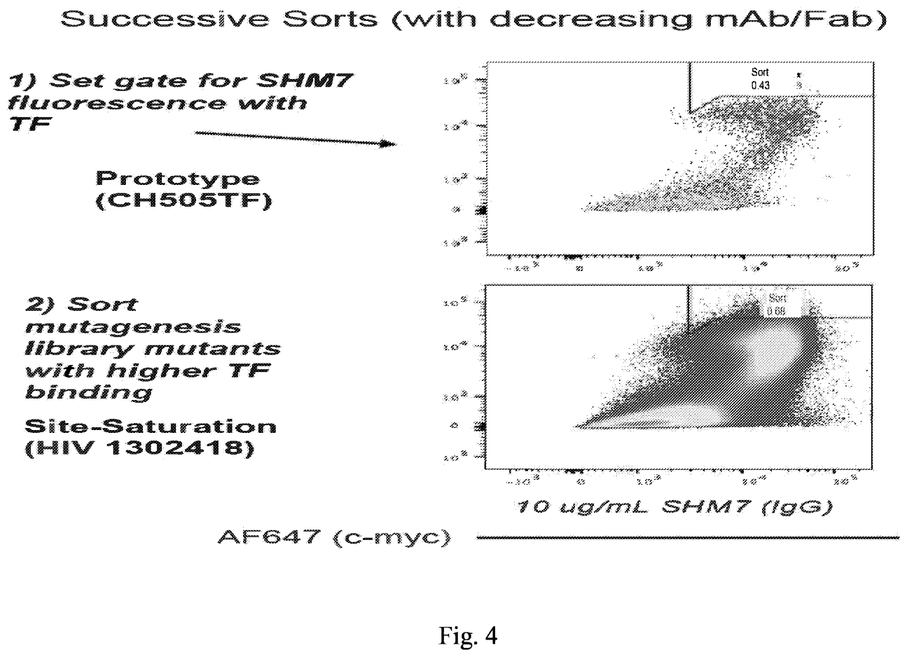

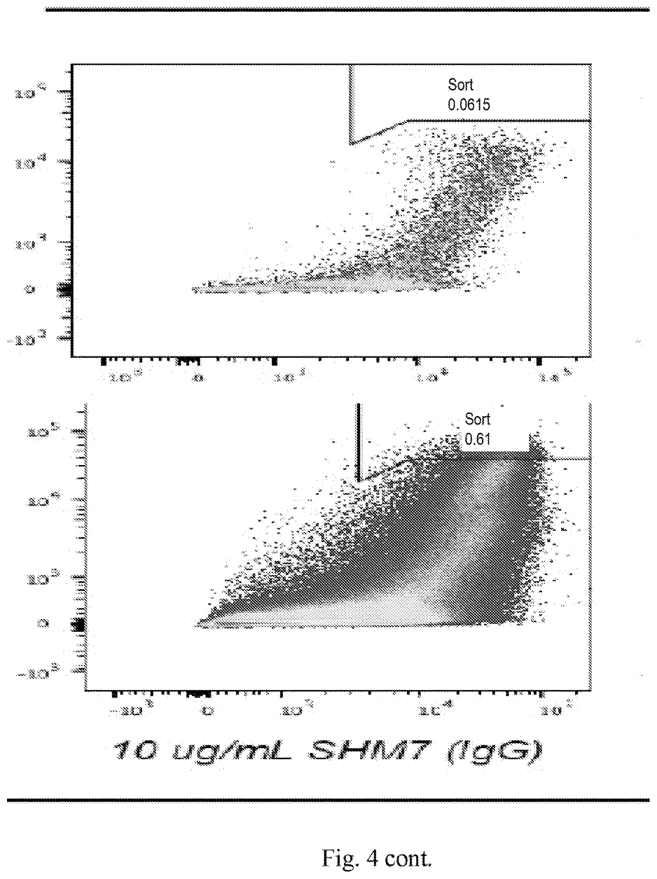

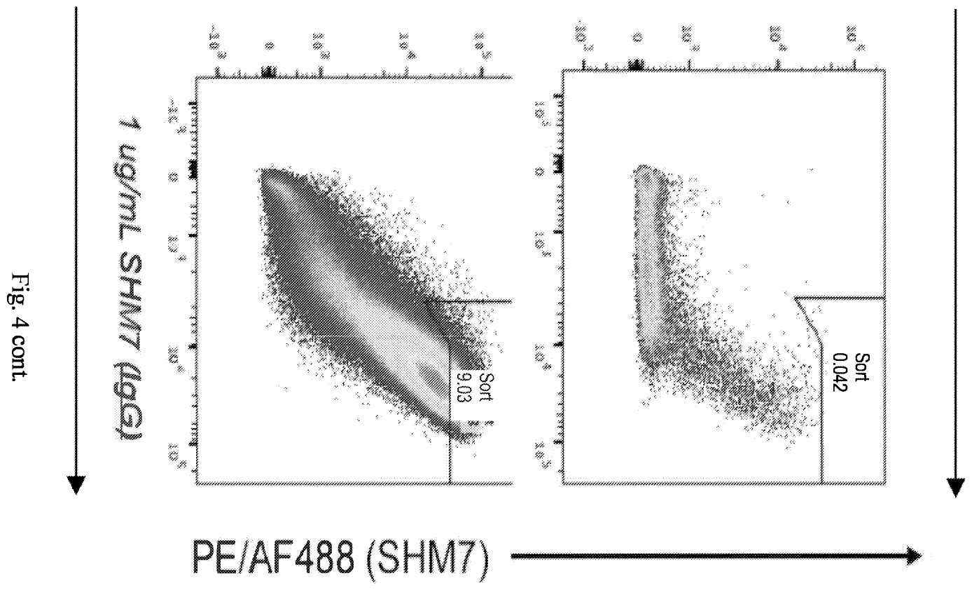

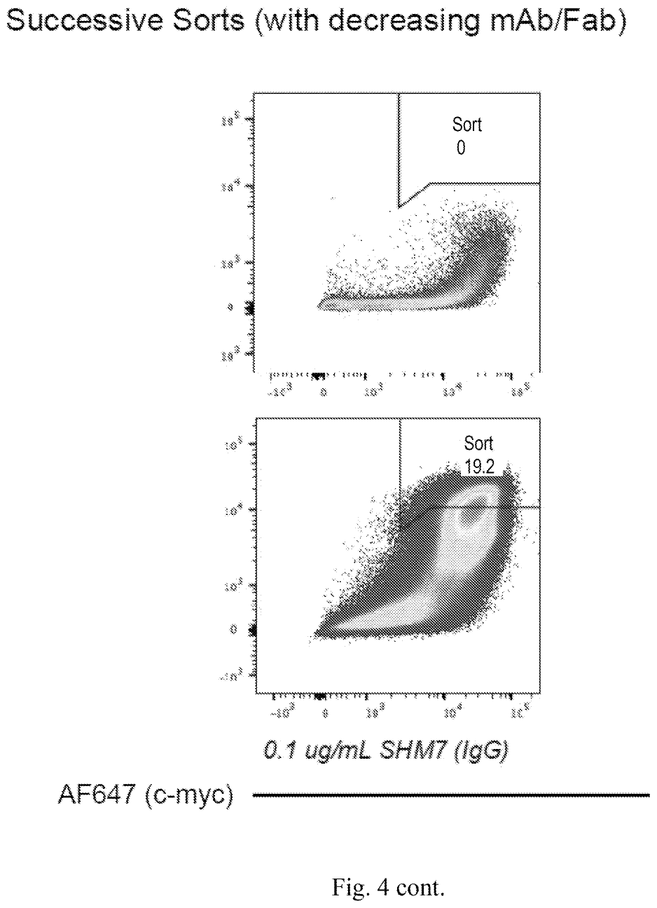

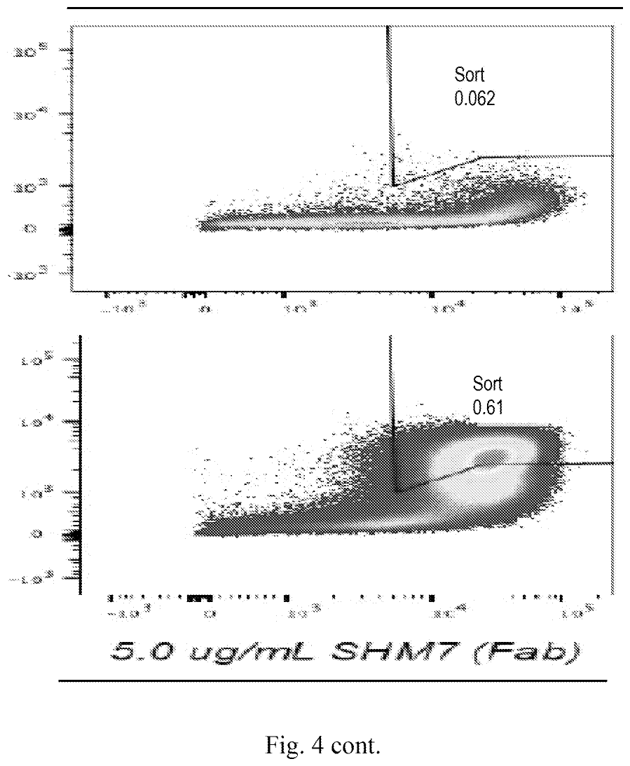

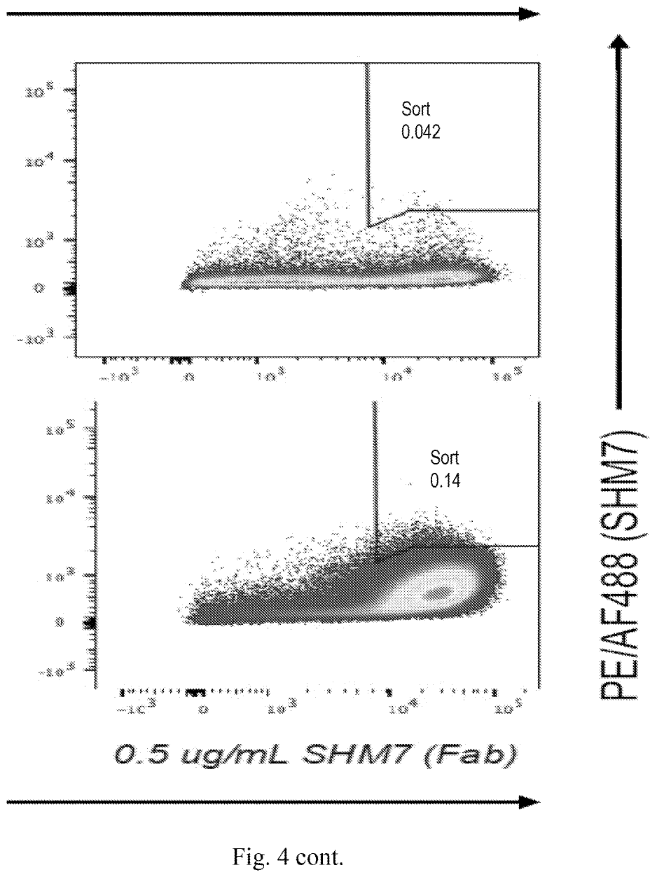

FIG. 4 shows successive enrichment of SHM 7 binding-enhanced mutants. A gate for SHM7 was set using fluorescence with TF. Then, the mutagenesis library mutants with higher TF binding were sorted.

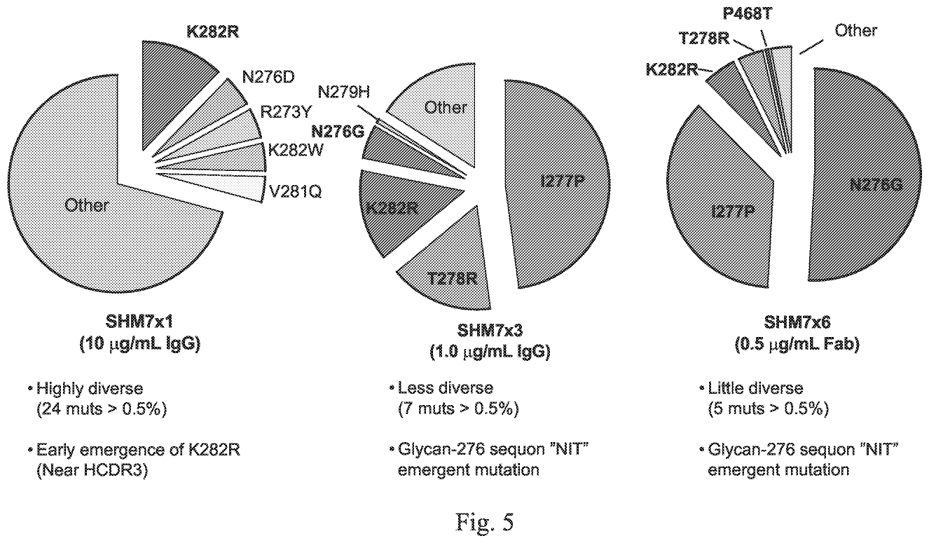

FIG. 5 shows the sequential selection of envelope variants enriched primarily D-loop mutants. SM-17 x1 showed highly diverse mutations (24 mutations >0.5%) and early emergence of K282R (near HCDR3). SMH7 x3 showed less diverse (7 mutations >0.5%) mutations and glycan-276 sequon “NIT” emergent mutation. SHM7 x6 showed little diverse (5 mutations >0.5%) and glycan-276 sequon “NIT” emergent mutation.

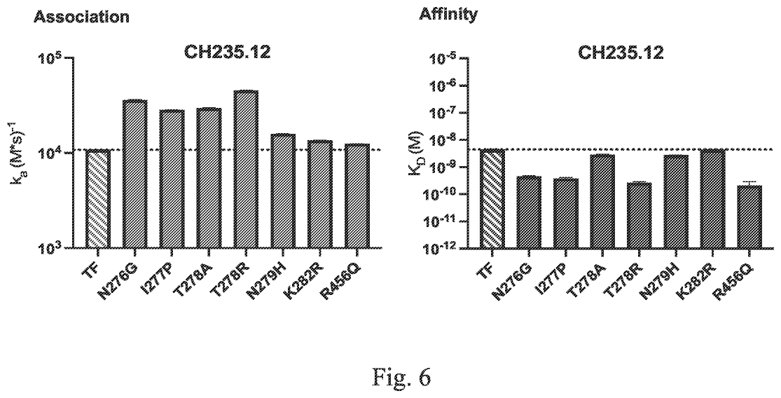

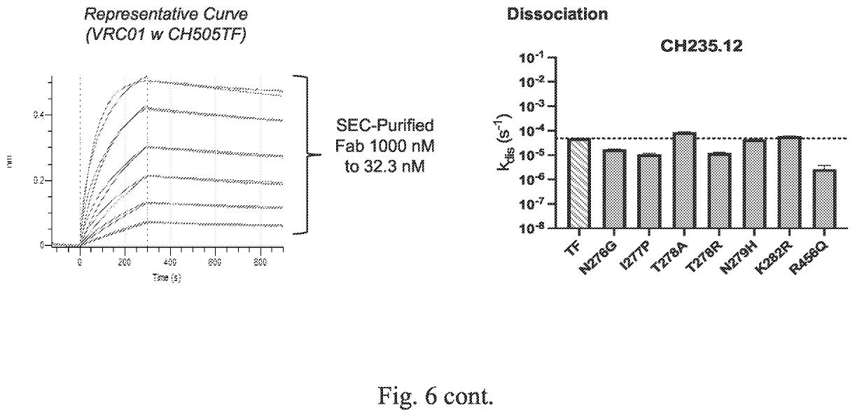

FIG. 6 shows the affinity of CH505 mutants by using bio-layer interferometry. It shows improvements in association rates for mature and early intermediates. There were also some improvements in dissociation for V7HI-46 antibodies.

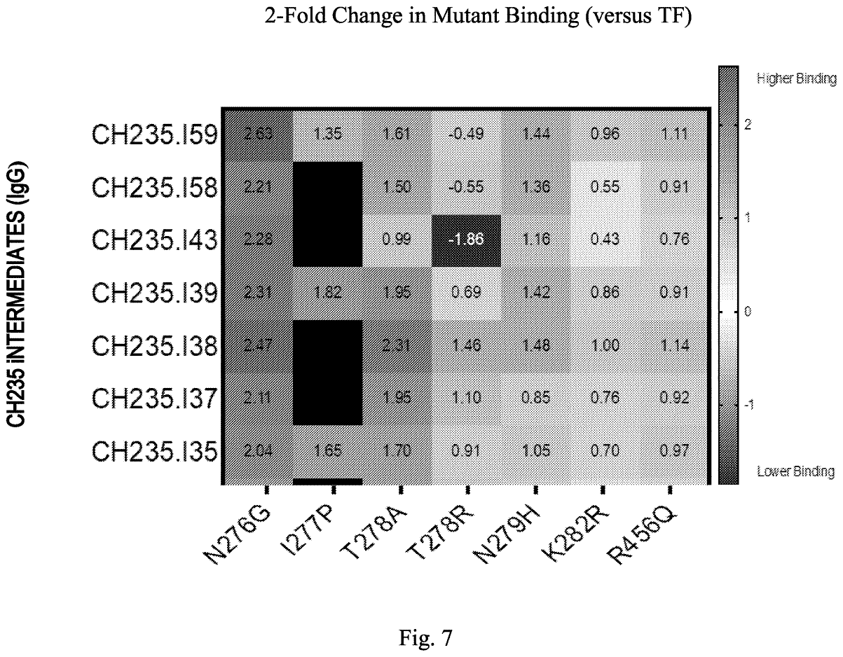

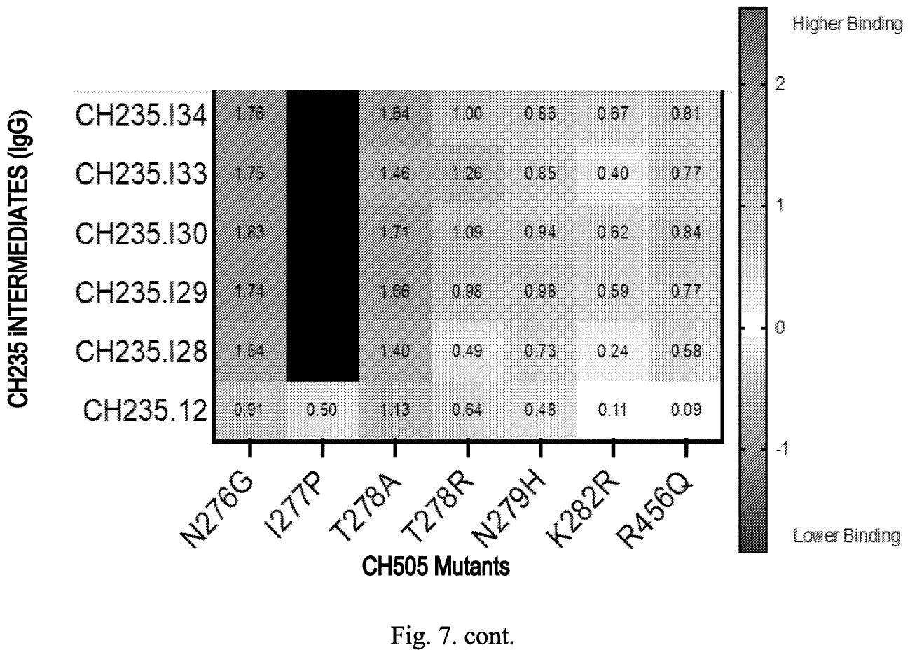

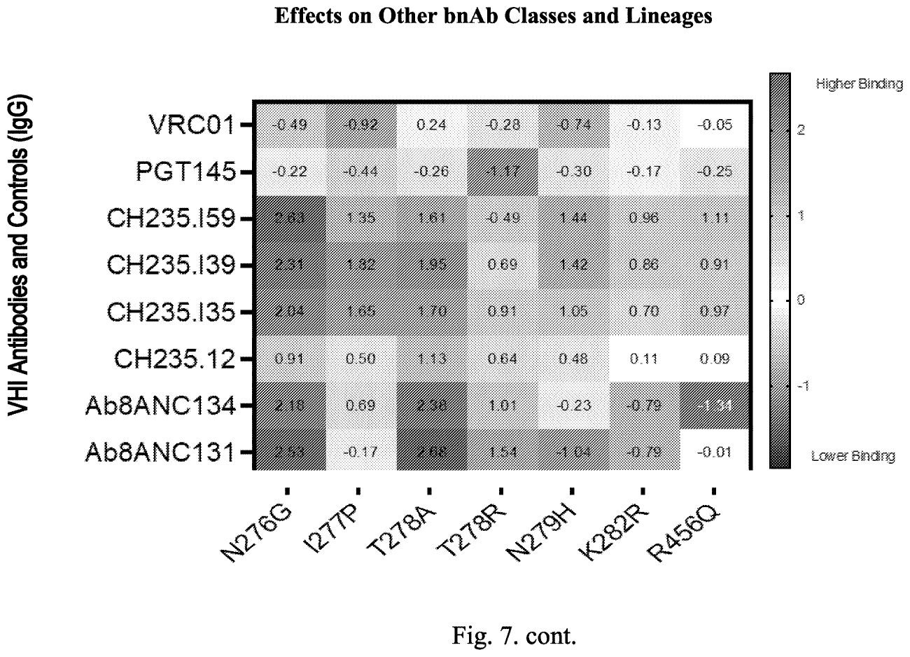

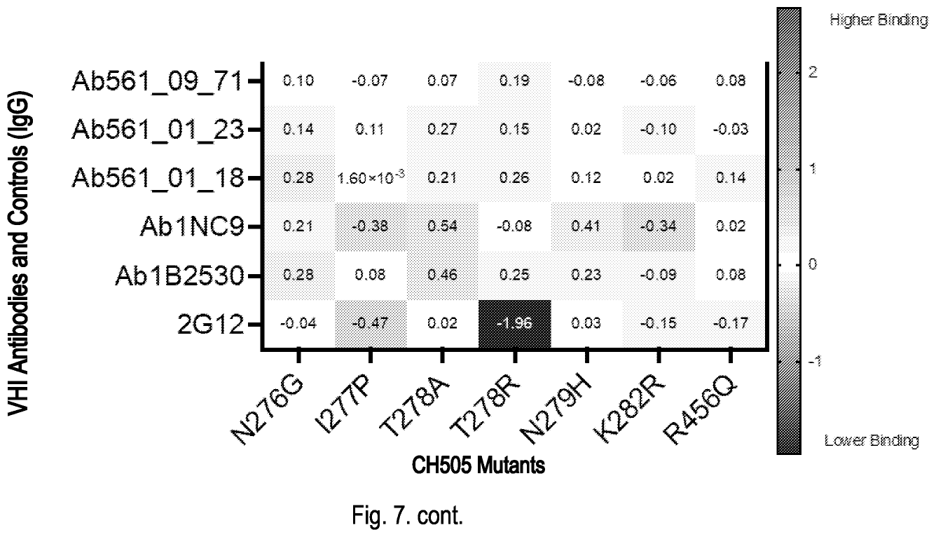

FIG. 7 shows that most mutations enhanced binding to CH235 lineage members.

In summary, removal of glycan-276 is antigenically dominant. This should be excluded in the future. T278R provides negative counter-selection against immature antibodies. N279H, K282R and R456Q provide modest improvements for CH235 lineage members (limited evidence for other VH1-46s). Combinations should be tested for synergism.

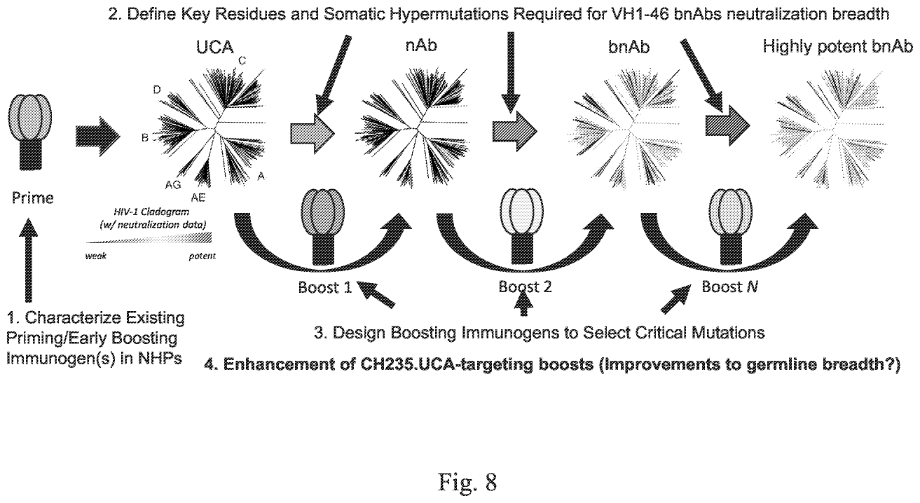

Next, the mutants were tested whether they show an enhancement of CH235.UCA-targeting boosts, resulting in improvements in germline breadth. See FIG. 8.

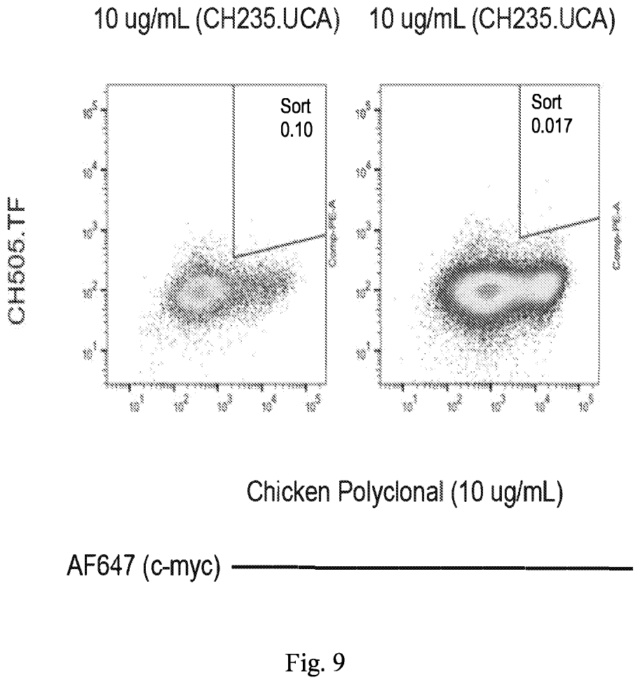

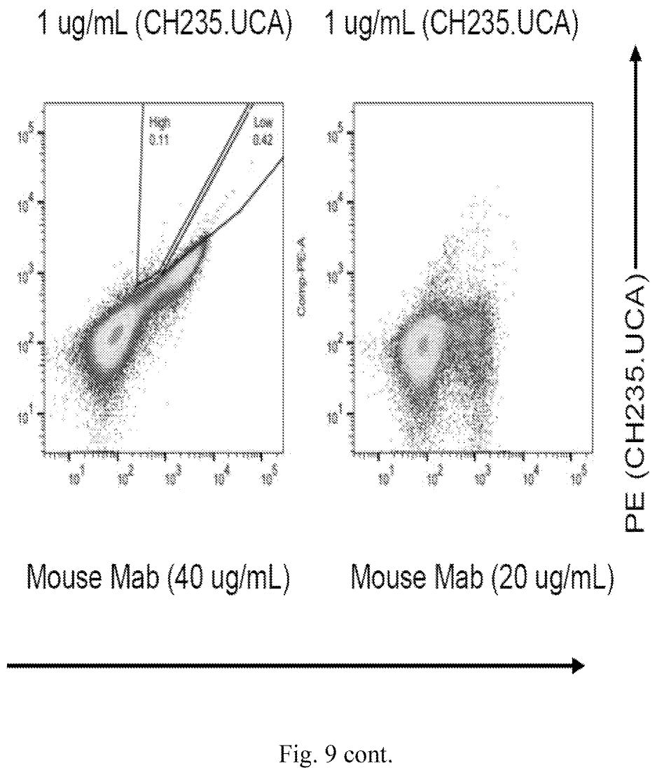

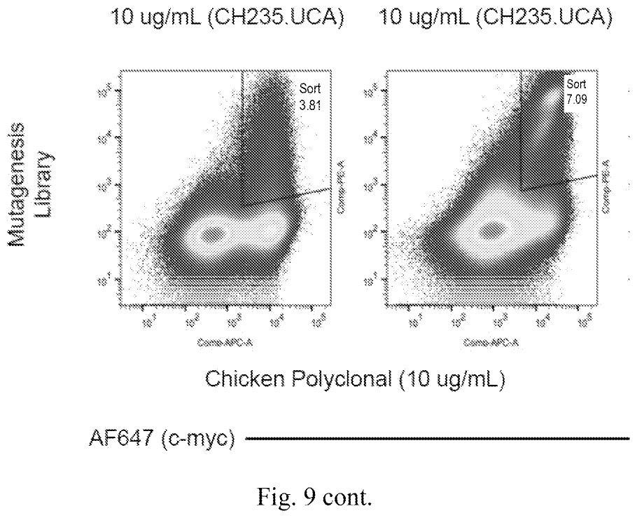

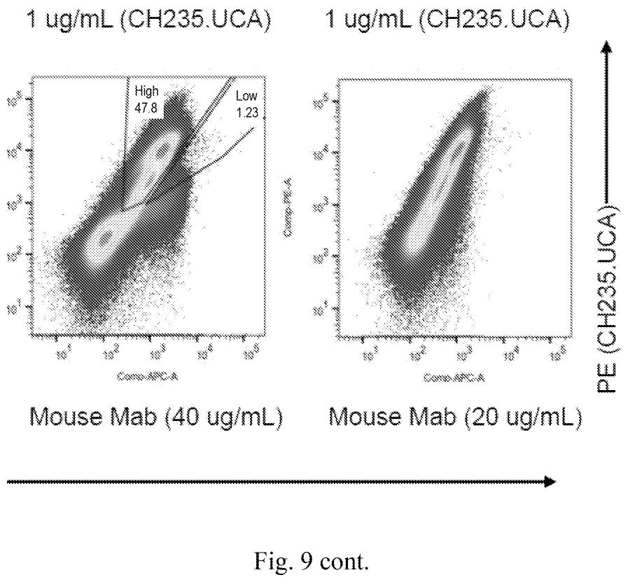

FIG. 9 shows successive enrichment of UCA binding-enhanced mutants.

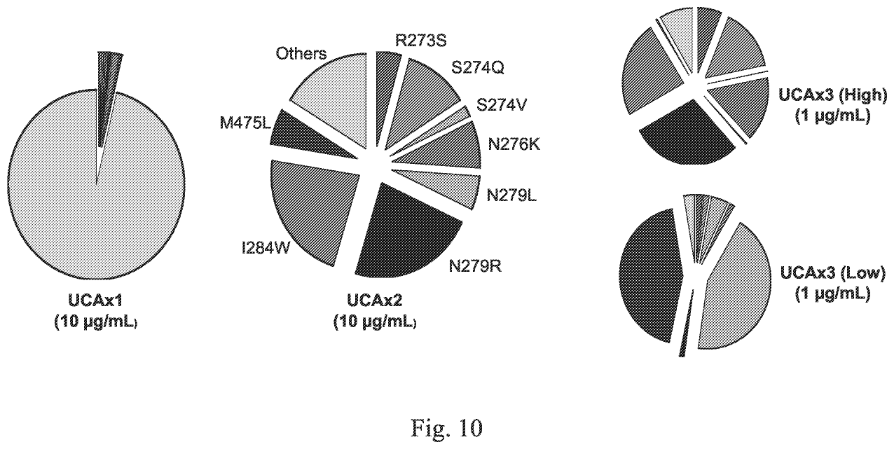

FIG. 10 shows that sequential selection of envelope variants again enriched primarily D-loop mutants.

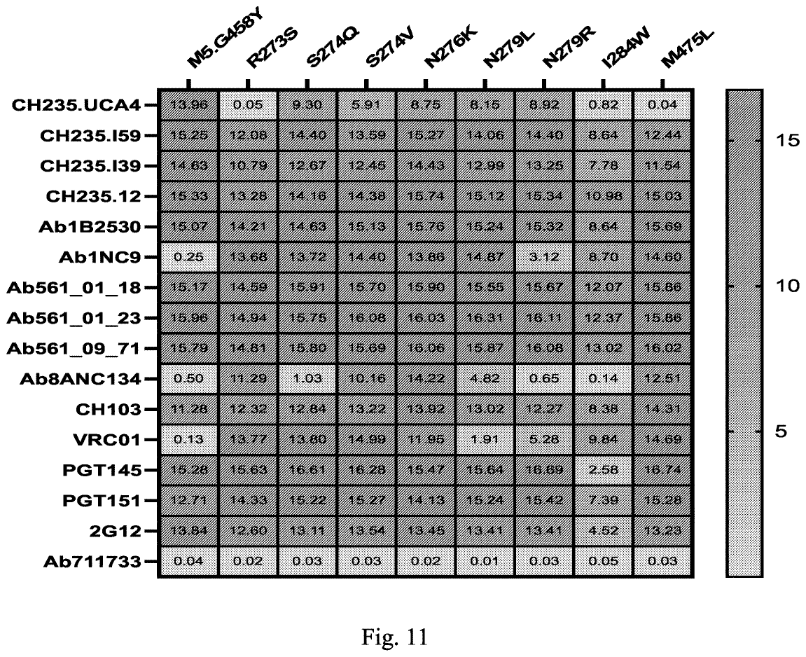

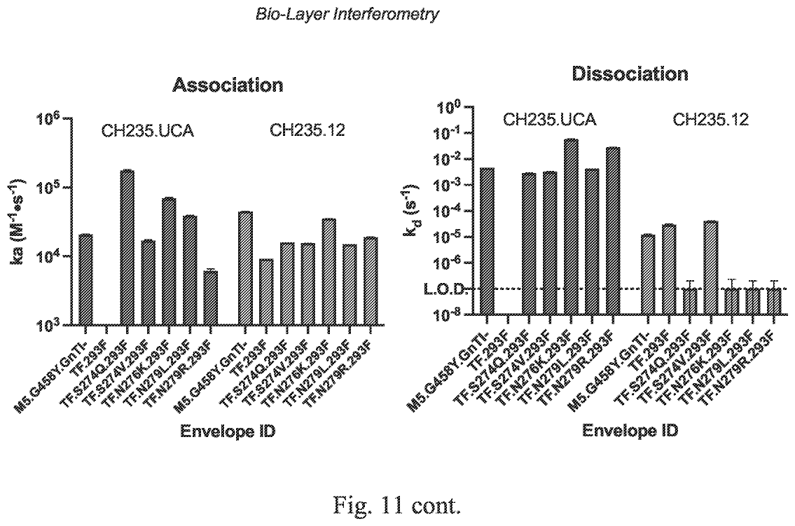

FIG. 11 shows ELISA and Bio-Layer Interferometry evaluation of C1-1235.UCA-sorted mutants.

In summary, loop D mutations are highly deterministic of CH235 antibody binding; glycan 276 knockout is not the dominant species: S274Q, N279R, 1284W; N279L and M475L clearly dominate lower affinity population; and a new “N2” Scanning Saturation Mutagenesis Library with two variable sites in defined subepitopes and invariant glycans was developed.

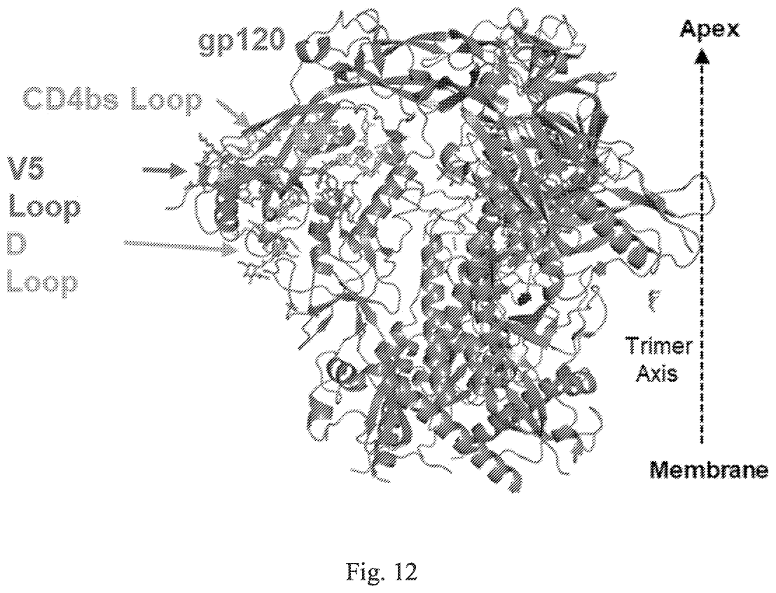

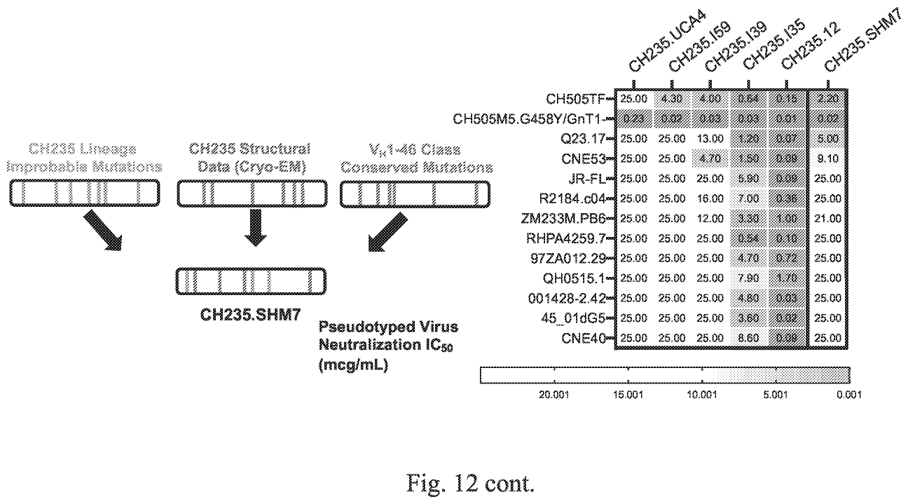

FIGS. 12-17 disclose evolving CD4 binding site immunogens via antibody lineage selection and mammalian cell display.

FIG. 12 shows a methodology. A site-scanning saturation library was generated by using saturation mutagenesis. There were 62 individual residues with 19 possible mutations and CH505TF chimera was used as a prototype. For selection, stable cell lines (293T) were selected using hygromycin. Surface expression was tested with a gene marker (c-myc). Fluorescence Activated Cell Sorting (FACS) was used to sort cells, and RNAs were extracted, followed by next generation sequencing (NGS) to determine enrichment,

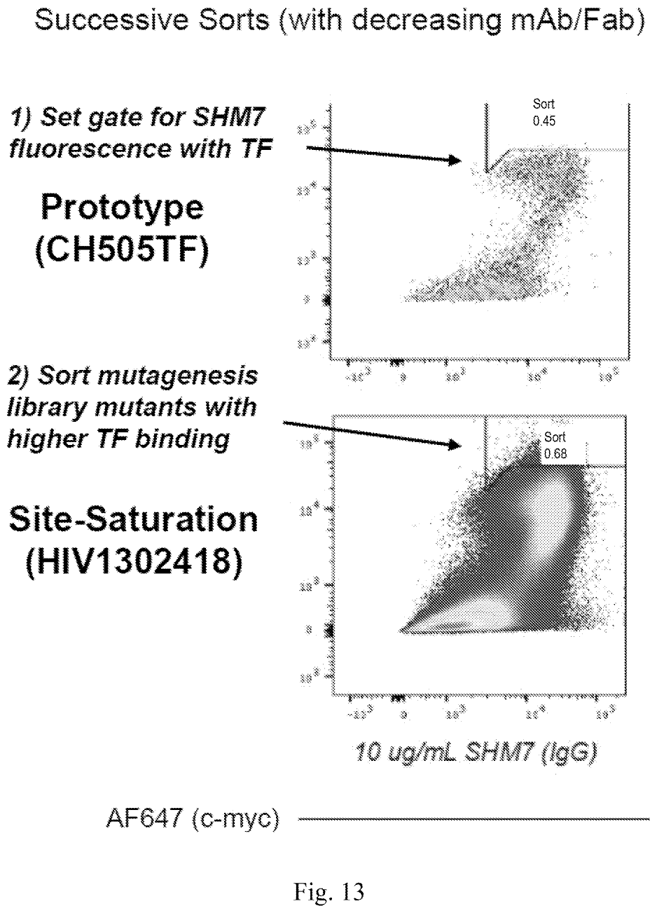

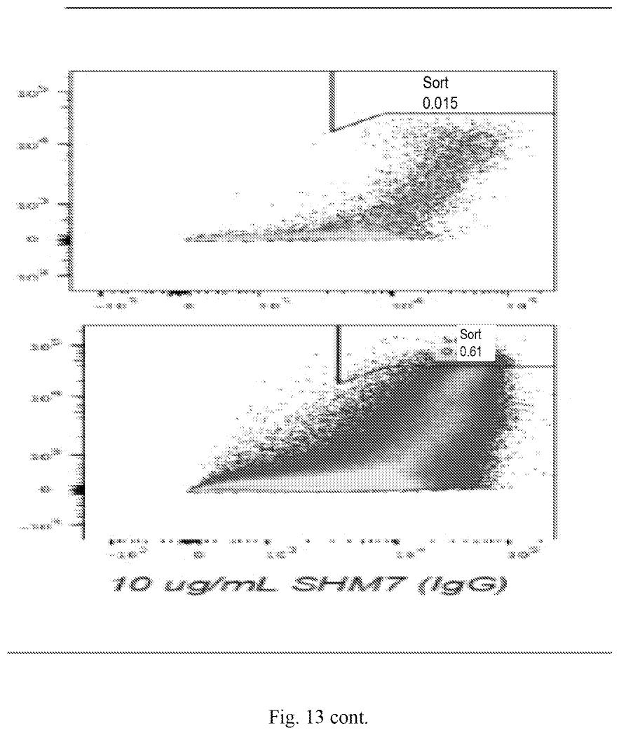

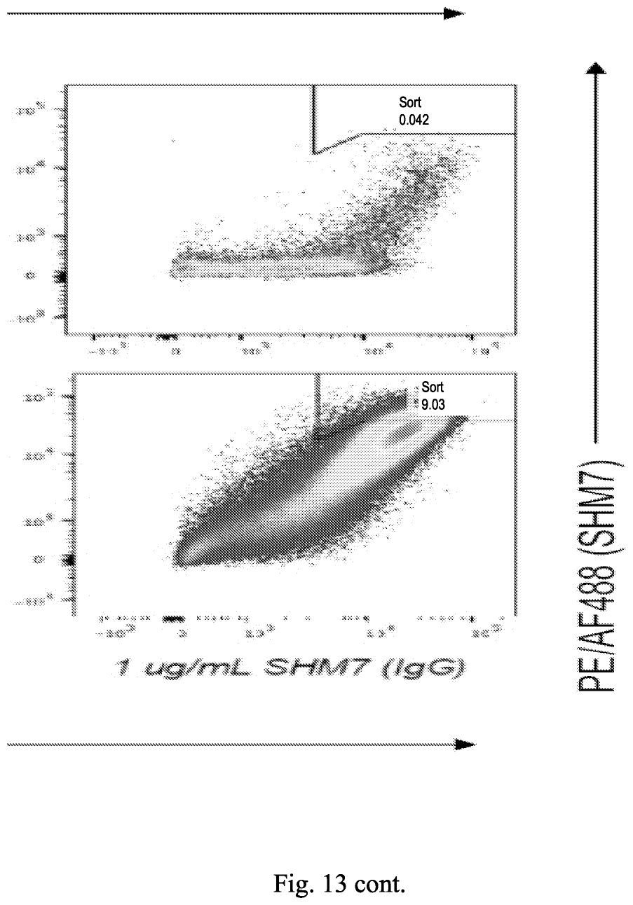

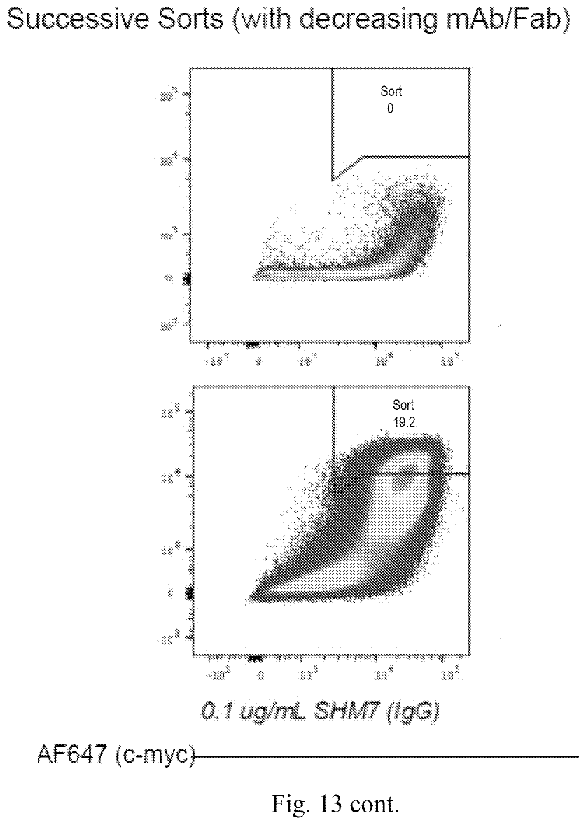

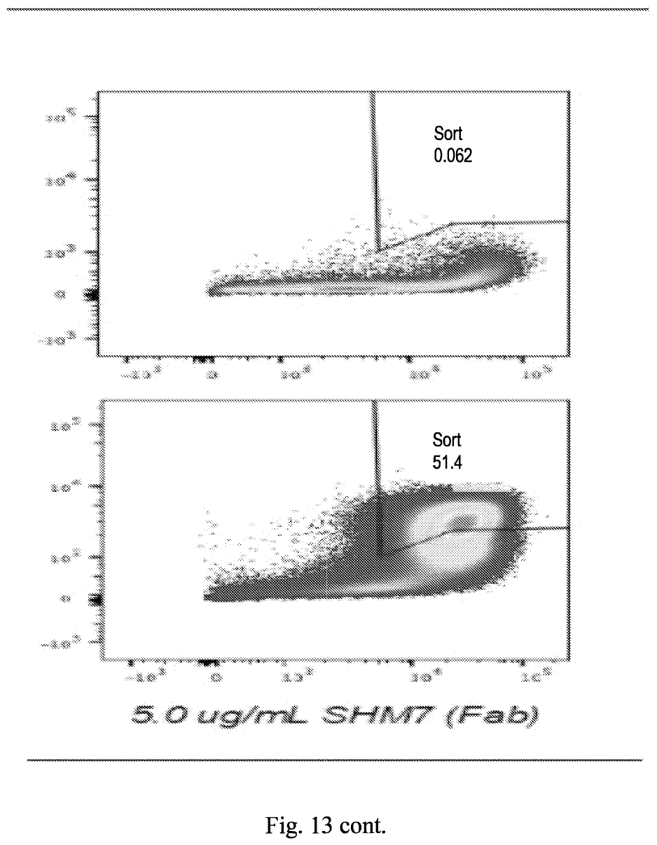

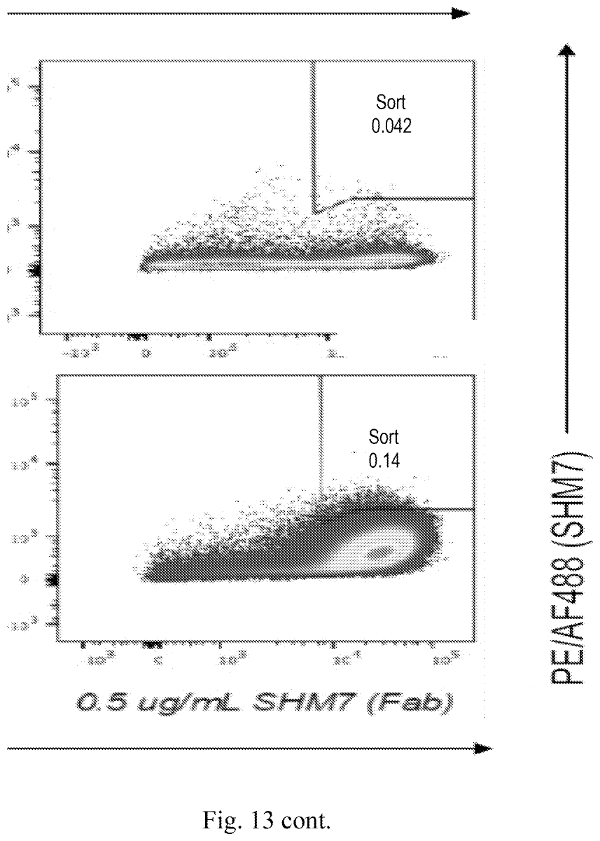

FIG. 13 shows the enrichment data of CH235.SHM7, where fluorescence with TF was used to set gate for SHM7. Mutagenesis library mutants with higher TF binding were sorted.

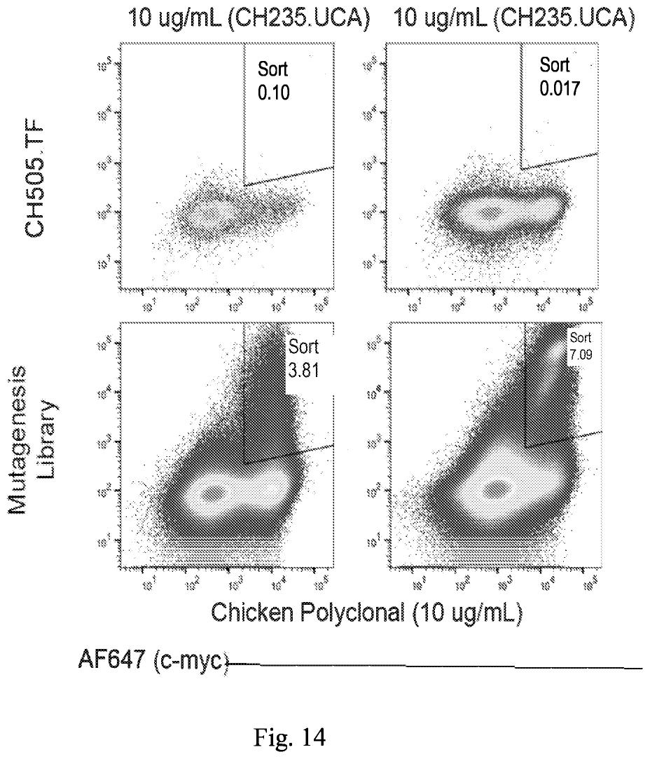

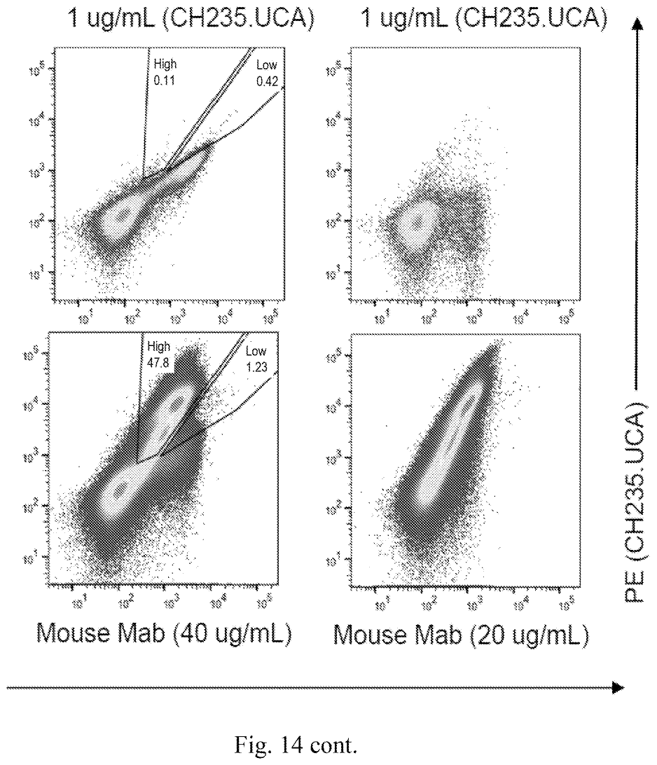

FIG. 14 shows the enrichment data of C1235.UCA.

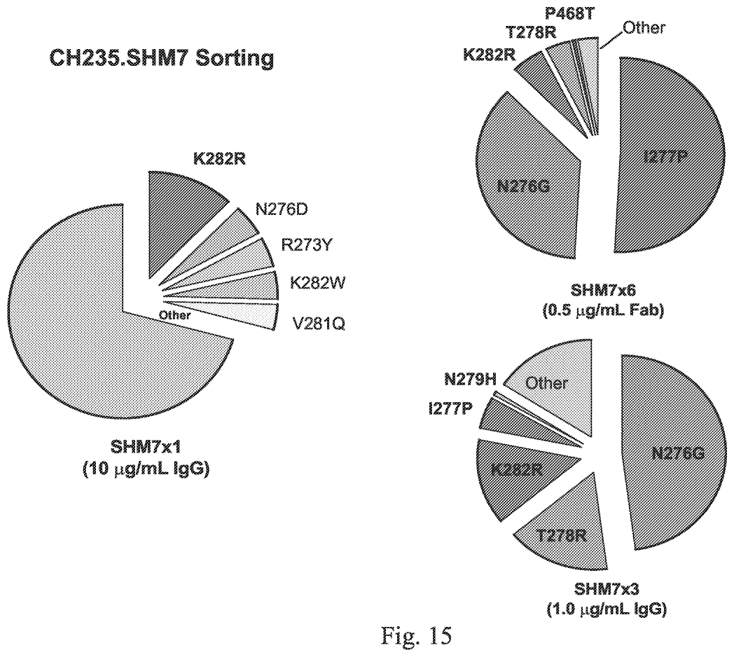

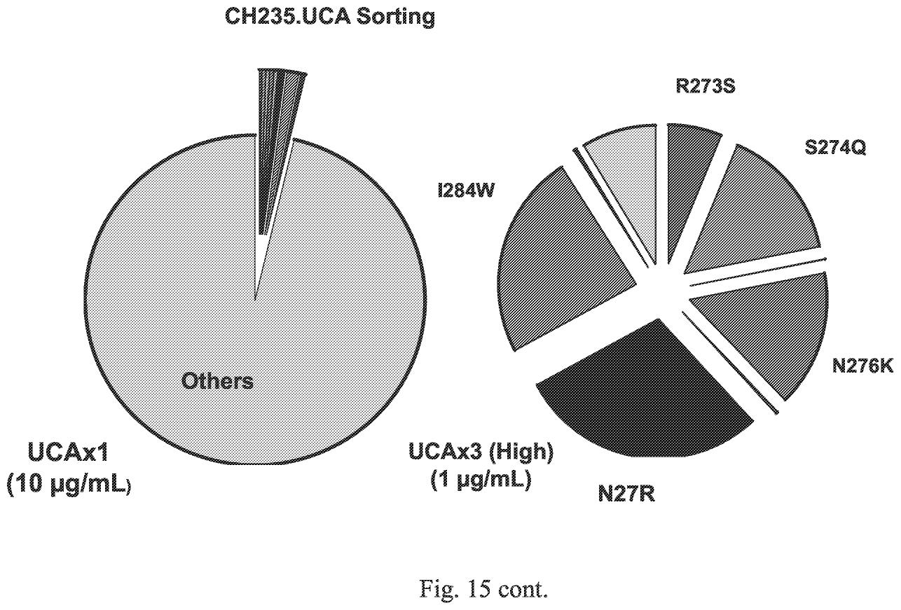

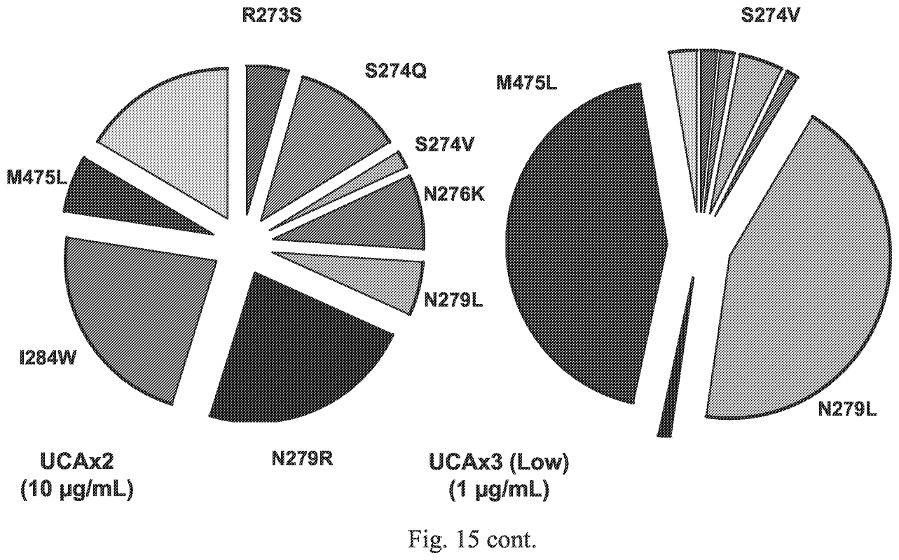

FIG. 15 shows the mutation enrichment data of CH235.SHM7 sorting and CH235.UCA sorting.

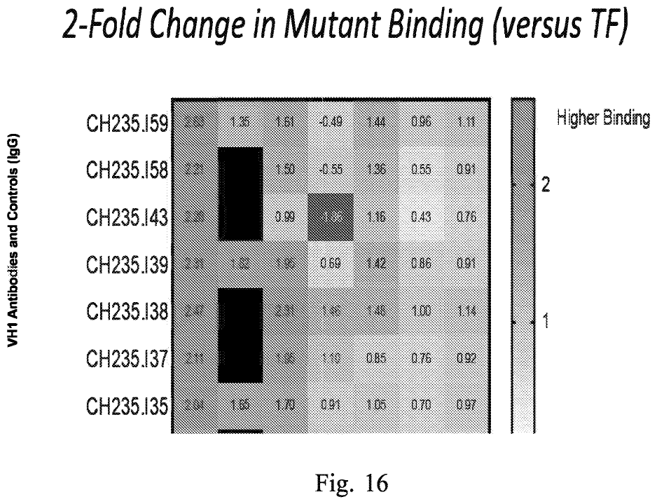

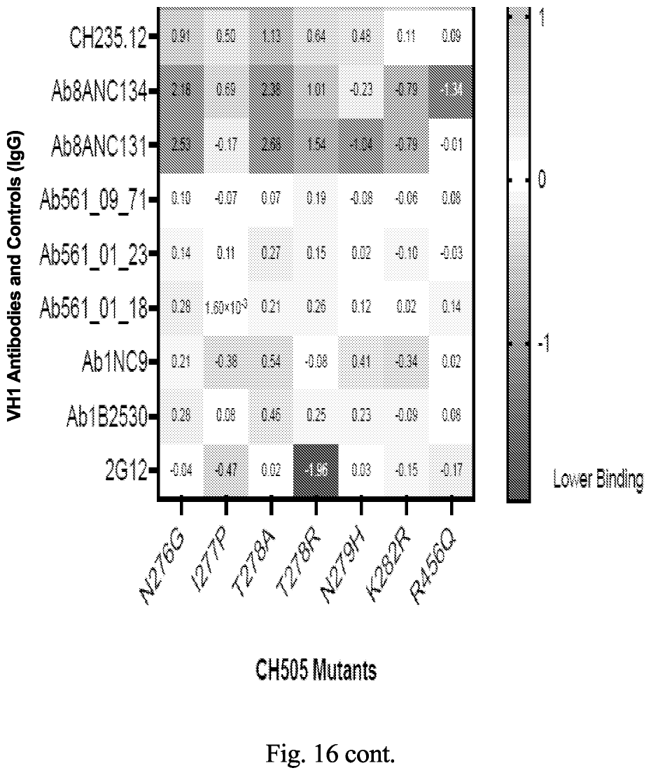

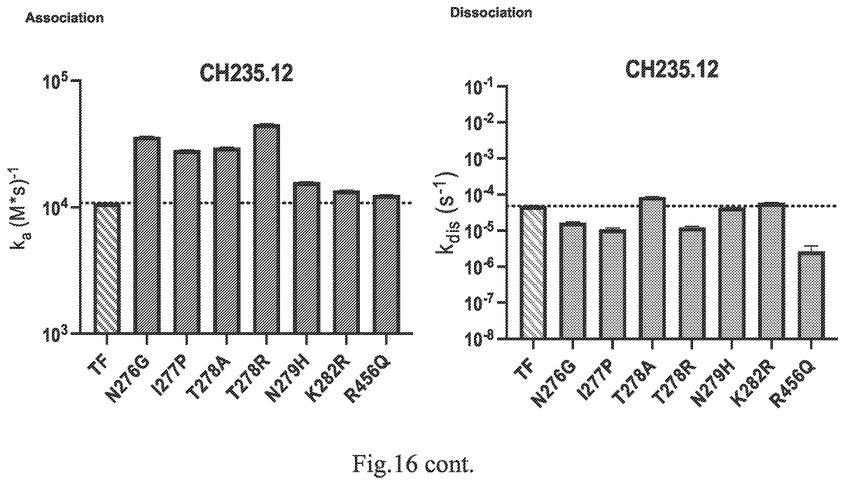

FIG. 16 shows the evaluation of C1235.SHM7-sorted mutants by using ELISA and Bio-Layer Interferometry.

FIG. 17A shows the evaluation of CH235.UCA-sorted mutants by using ELISA

FIG. 17B shows the kinetics of CH235.UCA-sorted mutants, CH235.12 showed a higher association rate and a lower dissociation rate than CH235UCA.

### Example 3

This example describes animal studies with HIV-1 envelopes designed to prime and boost CD4 binding antibodies lineages.

The envelopes described in Table 1, expressed as recombinant proteins are analyzed in animal studies including mouse and NHP animal models. The mouse animal model could be any model, including an animal model comprising a CH235 UCA transgene.

Any suitable adjuvant will be used.

The envelopes in Table 1 will be produced under cGMP conditions as a recombinant protein for use in Phase I clinical trial.

### Example 4

FIG. 19A shows that SHM7 heterologous reactivity requires a subset of mutations. The ELISA binding data demonstrate the role of different SHM7 mutations on heterologous envelope binding reactivity by single mutation reversion. SHM7 binding is presented as a comparison for each individual reversion. Scale is Log 10 area under the curve as measured by the trapezoid rule. The ELISA binding data shows that Tl 9K, L47W, W50I, D52N and R57S impacted the heterologous neutralization.

FIG. 19B shows that SHM7-selected double/triple mutants of CH505 place pressure on critical SHM7 mutations. The ELISA binding data demonstrate the role of different SHM7-selected mutations coupled in binding specific mutations from SHM7. SHM7 binding is presented as a comparison for each individual reversion and CH505.TF is presented as a comparison for each of the SHM7-selected mutant pairings. PGT145 is an envelope trimer apex bnAb that is a positive control for envelope reactivity and conformational integrity, while nCoV-NTD is an unrelated SARS-CoV-2 viral protein used as a negative control to ascertain binding specificity. Scale is in Log 10 area under the curve as measured via trapezoid rule.

FIG. 19C shows the continuation of FIG. 19B, showing difference in log 10 area under the curve binding for each SHM7 reversion, minus SHM7 log10area under the curve. This demonstrates that certain envelope mutant pairings may present selective pressure for specific mutations in SHM7, particularly those also deemed important for heterologous reactivity.

What is claimed is:

1. A recombinant HIV-1 envelope comprising all the consecutive amino acids between the signal peptide and avi-tag of SEQ ID NOs:10-24.

2. The recombinant HIV-1 envelope of claim 1, comprising all the consecutive amino acids between the signal peptide and avi-tag of SEQ ID NO: 14.

3. The recombinant HIV-1 envelope of claim 1, comprising all the consecutive amino acids between the signal peptide and avi-tag of SEQ ID NO: 15.

4. The recombinant HIV-1 envelope of claim 1, comprising all the consecutive amino acids between the signal peptide and avi-tag of SEQ ID NO: 16.

5. The recombinant HIV-1 envelope of claim 1, comprising all the consecutive amino acids between the signal peptide and avi-tag of SEQ ID NO: 10.

6. The recombinant HIV-1 envelope of claim 2, further comprising mutation K282R.

7. The recombinant HIV-1 envelope of claim 2, further comprising mutation R456Q.

8. The recombinant HIV-1 envelope of claim 3, further comprising mutation R456Q.

9. The recombinant HIV-1 envelope of claim 2, further comprising mutation K282R and R456Q.

10. The recombinant HIV-1 envelope of any one of claims 6-9, further comprising mutation N276G.

11. The recombinant HIV-1 envelope of any one of claims 1-10, wherein the envelope is a protomer comprised in a trimer.

12. A nucleic acid encoding the recombinant HIV-1 envelope of any one of claims 1-10.

13. The nucleic acid of claim 12, wherein the nucleic acid comprises the portion of SEQ ID NOs: 31-45 encoding all the consecutive amino acids between the signal peptide and avi-tag.

14. The nucleic acid of claim 13, wherein the nucleic acid sequence further comprises a portion encoding a signal peptide.

15. A recombinant trimer comprising three identical protomers of an HIV-1 envelope comprising all the consecutive amino acids between the signal peptide and avi-tag of SEQ ID NOs:10-24.

16. The recombinant trimer of claim 15, wherein the HIV-1 envelope comprises all the consecutive amino acids between the signal peptide and avi-tag of SEQ ID NO: 14.

17. The recombinant trimer of claim 15, wherein the HIV-1 envelope comprises all the consecutive amino acids between the signal peptide and avi-tag of SEQ ID NO: 15.

18. The recombinant trimer of claim 15, wherein the HIV-1 envelope comprises all the consecutive amino acids between the signal peptide and avi-tag of SEQ ID NO: 16.

19. The recombinant trimer of claim 15, wherein the HIV-1 envelope comprises all the consecutive amino acids between the signal peptide and avi-tag of SEQ ID NO: 10.

20. The recombinant trimer of claim 16, wherein the HIV-1 envelope further comprises mutation K282R.

21. The recombinant trimer of claim 16, wherein the HIV-1 envelope further comprises mutation R456Q.

22. The recombinant trimer of claim 17, wherein the HIV-1 envelope further comprises mutation R456Q.

23. The recombinant trimer of claim 16, wherein the HIV-1 envelope further comprises mutation K282R and R456Q.

24. The recombinant trimer of any one of claims 20-23, wherein the HIV-1 envelope further comprises mutation N276G.

25. An immunogenic composition comprising the recombinant trimer of any one of claims 15-24 and a carrier.

26. An immunogenic composition comprising the nucleic acid of any one of claims 12-14 and a carrier.

27. An immunogenic composition comprising the envelope of any one of claims 1-10 and a carrier.

28. The immunogenic composition of claims 25-27 further comprising an adjuvant.

29. The nucleic acid of any one of claims 12-14 or the immunogenic composition of claims 26, or 28 wherein the nucleic acid is operably linked to a promoter, and optionally wherein the nucleic acid is inserted in an expression vector.

30. A method of inducing an immune response in a subject comprising administering a composition comprising any suitable form of a nucleic acid(s) of any one of claims 12-14 or the envelope of any one of claims 1-10 in an amount sufficient to induce an immune response.

31. The method of claim 30, wherein the nucleic acid encodes a protomer of a SOSIP trimer.

32. The method of claim 30, wherein the envelope is a protomer of a SOSIP trimer.

33. The method of claim 305, wherein the composition further comprises an adjuvant.

34. The method of claim 30, further comprising administering an agent which modulates host immune tolerance.

35. The method of claim 30, wherein the envelope administered is multimerized in a liposome or nanoparticle.

36. The method of claim 30, further comprising administering one or more additional HIV-1 immunogens to induce a T cell response.

37. A composition comprising a nanoparticle and a carrier, wherein the nanoparticle comprises any one of the envelopes of claims 1-10.

38. The composition of claim 37, wherein the nanoparticle is ferritin self-assembling nanoparticle.

39. A composition comprising a nanoparticle and a carrier, wherein the nanoparticle comprises any one of the trimers of claims 11 or 15-24.

40. The composition of claim 39, wherein the nanoparticle is ferritin self-assembling nanoparticle.

41. The composition of claim 40, wherein the nanoparticle comprises multimers of trimers.

42. The composition of claim 40, wherein the nanoparticle comprises 1-8 trimers.

43. A composition comprising a nanoparticle and a carrier, wherein the nanoparticle comprises any one of the nucleic acids of claims 12-14.

44. The composition of claim 43, wherein the nanoparticle is a lipid nanoparticle.

45. The method of claim 30, wherein the composition is administered as a prime.

46. The method of claim 30, wherein the composition is administered as a boost.

47. A method of inducing an immune response in a subject comprising administering an immunogenic composition of any one of claims 25-28 in an amount sufficient to induce an immune response.

48. A composition comprising the recombinant trimer of any one of claims 15-24 and a carrier.

49. A composition comprising the nucleic acid of any one of claims 12-14 and a carrier.

50. A composition comprising the envelope of any one of claims 1-10 and a carrier.


SEQUENCE LISTING (46 sequences)

SEQ ID NO: 1 - protein, 24 aa, Homo sapiens
MPMGSLQPLATLYLLGMLVASVLA

SEQ ID NO: 2 - protein, 20 aa, synthetic construct
GGGGSGLNDIFEAQKIEWHE

SEQ ID NO: 3 - protein, 4 aa, Human immunodeficiency virus
REKR

SEQ ID NO: 4 - protein, 9 aa, Human immunodeficiency virus
RRVVEREKR

SEQ ID NO: 5 - protein, 9 aa, synthetic construct
ERVVEREKE

SEQ ID NO: 6 - protein, 4 aa, synthetic construct
SEKS

SEQ ID NO: 7 - protein, 79 aa, synthetic construct
  VARIANT 30..37: note = residues may be deleted
MRVMGIQRNYPQWWIWSMLGFWMLMICNGMWVTVYYGVPVWKEAKTTLFCASDAKAYEKEVHNVWATHACVPTDPNPQE

SEQ ID NO: 8 - protein, 5 aa, synthetic construct
LPXTG

SEQ ID NO: 9 - protein, 5 aa, synthetic construct
GGGGG

SEQ ID NO: 10 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSEGITNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 11 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENPTNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 12 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENIANNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 13 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENIRNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 14 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITHNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 15 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNVRTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 16 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTQDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 17 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIISSENITNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 18 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRQENITNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 19 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRVENITNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 20 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSEKITNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 21 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITLNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 22 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITRNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 23 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNVKTWIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 24 - protein, 667 aa, synthetic construct
MPMGSLQPLATLYLLGMLVASVLAAENLWVTVYYGVPVWKEAKTTLFCASDAKAYEKKVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNVKTIIVHLNESVKIECTRPNNKTRTSIRIGPGQWFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNLKDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGGSGLNDIFEAQKIEWHE

SEQ ID NO: 25 - protein, 11 aa, synthetic construct
  VARIANT 2: note = any amino acid
MXKDACLQSPV

SEQ ID NO: 26 - protein, 11 aa, synthetic construct
  VARIANT 3: note = any amino acid
MEXDALCQSPV

SEQ ID NO: 27 - protein, 11 aa, synthetic construct
  VARIANT 4: note = any amino acid
MEKXALCQSPV

SEQ ID NO: 28 - protein, 11 aa, synthetic construct
  VARIANT 5: note = any amino acid
MEKDXLCQSPV

SEQ ID NO: 29 - DNA, 72 bp, synthetic construct
  source 1..72: mol_type = other DNA
atgcccatgggcagcctgcagcccctggccaccctgtacctgctgggcatgctggtggctagcgtgctggcc

SEQ ID NO: 30 - DNA, 78 bp, synthetic construct
  source 1..78: mol_type = other DNA
ggcggaggcctggtgcctcagcagagcggcggcctgaacgacatcttcgaggcccagaagatcgagtggcacgaaggc

SEQ ID NO: 31 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagggcatcacaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 32 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaaccccacaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 33 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcgcaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 34 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcagaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 35 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcacacataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 36 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcacaaataatgtgaggactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 37 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcacaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacacaagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 38 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatcagcagcgagaacatcacaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 39 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgccaggagaacatcacaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 40 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcgtcgagaacatcacaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 41 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaagatcacaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 42 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcacactgaatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 43 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcacaagaaatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 44 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcacaaataatgtgaagacttggatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacatgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 45 - DNA, 2022 bp, synthetic construct
  source 1..2022: mol_type = other DNA
atgcccatgggcagcctgcagcccctggccaccctgtacctgctgggcatgctggtggctagcgtgctggccgccgagaacctgtgggtgaccgtctactatggcgtgcccgtctggaaggaagccaaaaccacactgttctgcgctagcgacgctaaggcatacgagaaaaaagtgcacaatgtctgggctactcatgcatgcgtgcctaccgatccaaatccccaggagatggtgctgaagaacgtcacagaaaactttaatatgtggaagaacgacatggtggatcagatgcacgaggacgtgatcagcctgtgggatcagtccctgaagccatgcgtgaaactgactcccctgtgcgtcaccctgaactgtactaatgccaccgcttccaacagctccatcattgaggggatgaagaactgttctttcaatatcactaccgagctgcgcgacaagcgagaaaagaaaaatgccctgttttacaaactggacatcgtgcagctggatggcaactctagtcagtatagactgattaactgcaatacaagcgtgatcactcaggcatgtccaaaggtcagtttcgatcctattccaatccactactgcgcacccgccggatatgctatcctgaagtgtaacaacaagaccttcaccggcactgggccttgcaacaacgtgagcaccgtccagtgtacacatggcattaagccagtggtcagcacccagctgctgctgaacggcagcctggcagagggcgaaatcattatccgcagcgagaacatcacaaataatgtgaagactatcatcgtccacctgaacgagagcgtgaagattgaatgcacacggcccaacaacaagaccaggacatccattcgcatcggacctggccagtggttctacgctactggccaggtcatcggggacatcagagaggcctattgtaacatcaatgagtcaaagtggaatgaaactctgcagagggtgagcaagaaactgaaggaatacttccctcacaaaaacatcacctttcagccatcaagcggcggggacctggagattacaactcattctttcaattgcggaggcgaattcttttactgtaacacctcctctctgtttaatcgcacatatatggctaacagtactgatatggcaaactctactgagaccaatagtacacgaactattaccatccattgccggatcaagcagattatcaacatgtggcaggaagtggggcgggccatgtatgctccccctattgcaggaaatattacctgtatcagcaacattaccggcctgctgctgacaagagacgggggaaagaacaatacagagacttttaggcctggcgggggaaacctgaaagataattggcgctccgagctgtacaagtataaagtggtcaagatcgaaccactgggagtggcacctacccgatgtaaacggagagtggtcggaaggcgccgacggagaagggcagtgggaatcggagccgtcttcctgggctttctgggagcagctggcagcacaatgggagcagcctctatgaccctgacagtgcaggctcgaaatctgctgagtgggatcgtgcagcagcagtcaaacctgctgcgagcaccagaggcacagcagcatctgctgaagctgaccgtgtggggcatcaagcagctgcaggccagagtgctggctgtcgaacggtacctgagagatcagcagctgctgggaatctggggatgcagcggaaagctgatttgctgtacaaacgtgccctggaatagttcatggtcaaacaggaatctgagcgagatctgggacaatatgacctggctgcagtgggataaggaaatcagtaactacacacagatcatctatggcctgctggaggaatcacagaaccagcaggagaaaaatgaacaggacctgctggccctggatggcggaggcctggtgcctcagcagagcggcggcctgaacgacatcttcgaggcccagaagatcgagtggcacgaaggctga

SEQ ID NO: 46 - protein, 6 aa, synthetic construct
  VARIANT 3: note = any amino acid but most commonly Ala, Ser or Glu
LPXTGG